(19) United States
(12) Patent Application Publication
(10) Pub. No.: US 2025/0325682 A1
(43) Pub. Date: Oct. 23, 2025

(54) FKBP52-DERIVED THERAPEUTIC PEPTIDES THAT INHIBIT PATHOLOGICAL TAU AGGREGATION FOR THERAPEUTIC PURPOSES

(71) Applicant: ASSOCIATION INSTITUT PROFESSEUR BAULIEU, Le Kremlin-Bicêtre Cedex (FR)

(72) Inventors: Etiene-Emile BAULIEU, Paris (FR); Béatrice CHAMBRAUD, Paris (FR); Julien GIUSTINIANI, Paris (FR); Omar DOUNANE, Cachan (FR); Cillian BYRNE, Paris (FR)

(73) Assignee: ASSOCIATION INSTITUT PROFESSEUR BAULIEU, Le Kremlin-Bicêtre Cedex (FR)

(21) Appl. No.: 18/642,615

(22) Filed: Apr. 22, 2024

(51) Int. Cl. A61K 47/64; C12N 15/86; G01N 33/50; G01N 33/58

(52) U.S. Cl. CPC A61K 47/64; C12N 15/86; G01N 33/5058; G01N 33/58; C12N 2740/15043; C12N 2750/14143; G01N 2333/99

(57) ABSTRACT

The invention is directed to peptide fragments of FKBP52 that inhibit Tau protein aggregation and ameliorate tauopathies like Alzheimer's Disease (AD). It also involves modifications to these peptides to improve their pharmacokinetic and pharmacodynamic properties.

### REFERENCE TO A SEQUENCE LISTING

In accordance with 37 CFR § 1.833-1835 and 37 CFR § 1.77 (b) (5), the specification makes reference to a Sequence Listing submitted electronically as a .xml file named “552944US_062424_ST26.xml”. The .xml file was generated on Jun. 24, 2024 and is 12,575 bytes in size. The entire contents of the Sequence Listing are hereby incorporated by reference.

### FIELD OF THE INVENTION

The invention relates to the fields of peptide chemistry and medicine including to peptide-based neuroprotection and treatment of tauopathies using specific peptides derived from FK506 binding protein 52 identified herein as FKBP52.

### BACKGROUND OF THE INVENTION

A large number of neurodegenerative diseases, including Alzheimer Disease (AD), Pick's disease (PiD), progressive supranuclear palsy (PSP), corticobasal degeneration (CBD), and frontotemporal lobar degeneration due to the mutation of Tau gene (FTLD-Tau) are characterized by intra-neuronal aggregates of Tau, which are hallmarks of those disorders of the human brain now called tauopathies. In FTLD-Tau, different Tau mutations have been described (i.e., Tau-P301S, Tau-P301L) and about half of the known mutations show effects at the level of the Tau protein, reducing its normal function and increasing its propensity to assemble into abnormal filaments; Goedert M, Tau gene mutations and their effects. MOV DISORD, 2005, 20 Suppl 12, S45-52. Tau proteins are widely expressed in the central nervous system, predominantly in neurons where they play a key role in regulating microtubule dynamics, axonal transport and neurite outgrowth; Weingarten M D, Lockwood A H, Hwo S Y, Kirschner M W (1975) A protein factor essential for microtubule assembly. Proc Natl Acad Sci USA 72, 1858-1862. Avila J, Lucas J J, Perez M, Hernandez F, Role of tau protein in both physiological and pathological conditions. PHYSIOL. REV, 2004, 84, 361-384. Alternative splicing of Tau results in six isoforms present in the adult brain that differ in their sizes and in their effects on microtubular dynamics; Goedert M, Jakes R (1990) Expression of separate isoforms of human tau protein: correlation with the tau pattern in brain and effects on tubulin polymerization. EMBO J 9, 4225-4230. Tau is a natively unfolded protein; its aggregation is a multistep process converting a soluble and monomeric Tau form into an insoluble, hyperphosphorylated and filamentous form; Barghorn S, Mandelkow E (2002) Toward a unified scheme for the aggregation of tau into Alzheimer paired helical filaments. BIOCHEMISTRY 41, 14885-14896.

During this process, transient small oligomeric species do form filaments that specifically present a twist appearance in AD brains and are the major source of higher Tau aggregates called NeuroFibrillary Tangles (NFTs). Growing evidence suggest that NFTs do not appear to be the main toxic entities leading to disease but it is rather the intermediate entity, such as oligomeric soluble forms of Tau, that could be responsible for toxicity and disease; Santacruz K, Lewis J. Spires T, Paulson J, Kotilinek L, Ingelsson M. Guimaraes A, DeTure M, Ramsden M, McGowan E, Forster C, Yue M, Ore J, Janus C, Mariash A, Kuskowski M, Hyman B. Hutton M, Ashe K H, Tau suppression in a neurodegenerative mouse model improves memory function. SCIENCE 2005, 309, 476-481. Huang Y, Wu Z, Zhou B,) Behind the curtain of tauopathy: a show of multiple players orchestrating tau toxicity. CELL MOL LIFE SCI 2016, 73, 1-21.

The inventors have previously discovered that the protein named FKBP52 interacts physically and functionally with Tau showing an antagonist effect of FKBP52 on Tau's tubulin assembly; Chambraud B, Sardin E, Giustiniani J, Dounane O, Schumacher M, Goedert M, Baulieu E E, A role for FKBP52 in Tau protein function. PROC NATL ACAD SCI USA, 2010 107, 2658-2663. The inventors considered that FKBP52 might represent a promising target, leading to innovative therapeutic attempts centered on Tau. FKBPs (FK506 Binding Proteins) are a family of ubiquitously expressed protein folding chaperones with a peptidyl prolyl cis/trans isomerase (PPiase) activity. The FKBPs differ by the structure of domains and show different subcellular localizations, strongly suggesting specific functions for each FKBP; Gothel S F, Marahiel M A (1999) Peptidyl-prolyl cis-trans isomerases, a superfamily of ubiquitous folding catalysts. Cell Mol Life Sci 55, 423-436. These proteins are particularly abundant in the nervous system and involved in neurodegenerative disorders; Chattopadhaya S, Harikishore A, Yoon H S (2011) Role of FK506 binding proteins in neurodegenerative disorders. Curr Med Chem 18, 5380-5397. FKBP52 (FKBP of MW 52 kDa) was first cloned in our laboratory and its 3D structure revealed a modular organization with four functional domains; Chattopadhaya S, Harikishore A, Yoon H S (2011) Role of FK506 binding proteins in neurodegenerative disorders. Curr Med Chem 18, 5380-5397.

Two consecutive FKBP domains have been determined (FK1 AA 31-139, FK2 AA 149-267). While the second FK2 domain shares 34% of identity with FK1, only FK1 presents peptidyl-prolyl isomerase (PPlase) activity; Chambraud B, Rouviere-Fourmy N, Radanyi C, Hsiao K, Peattie D A, Livingston D J, Baulieu E E (1993) Overexpression of p59-HBI (FKBP59), full length and domains, and characterization of PPlase activity. Biochem Biophys Res Commun 196, 160-166. The C-Terminal part of FKBP52 contains additional functional domains such as a tetratricopeptide repeat domain that displays a co-chaperon activity and which serves as binding site for molecular chaperone HSP90; Bose S, Weikl T, Bugl H, Buchner J (1996) Chaperone function of Hsp90-associated proteins. Science 274, 1715-1717. Radanyi C, Chambraud B, Baulieu E E (1994) The ability of the immunophilin FKBP59-HBI to interact with the 90-kDa heat shock protein is encoded by its tetratricopeptide repeat domain. Proc Natl Acad Sci USA 91, 11197-11201.

Finally an α-helix in its extreme C-terminus includes a putative calmodulin binding site; Massol N, Lebeau M C, Renoir J M, Faber L E, Baulieu E E (1992) Rabbit FKBP59-heat shock protein binding immunophillin (HBI) is a calmodulin binding protein. Biochem Biophys Res Commun 187, 1330-1335. FKBP52 protein expression is strongly decreased in the frontal cortex of AD and FTLD-Tau brains and this decrease is highly correlated with the accumulation and aggregation of pathological Tau; Giustiniani J, Sineus M, Sardin E, Dounane O, Panchal M, Sazdovitch V, Duyckaerts C, Chambraud B. Baulieu E E (2012) Decrease of the immunophilin FKBP52 accumulation in human brains of Alzheimer's disease and FTDP-17. J Alzheimers Dis 29, 471-483. The inventors previously reported that FKBP52 is able to induce Tau oligomerization depending of the nature of Tau suggesting an involvement of FKBP52 in Tau aggregation; Giustiniani J, Guillemeau K, Dounane O, Sardin E, Huvent I, Schmitt A, Hamdane M, Buee L, Landrieu 1, Lippens G, Baulieu E E, Chambraud B (2015) The FK506-binding protein FKBP52 in vitro induces aggregation of truncated Tau forms with prion-like behavior. FASEB J. Giustiniani J, Chambraud B, Sardin E, Dounane O, Guillemeau K, Nakatani H, Paquet D, Kamah A, Landrieu I, Lippens G, Baulieu E E, Tawk M (2014) Immunophilin FKBP52 induces Tau-P301L filamentous assembly in vitro and modulates its activity in a model of tauopathy. Proc Natl Acad Sci USA 111, 4584-4589. Recently, the inventors have increased their knowledge of Tau-FKBP52 interaction using different biochemical and biophysical approaches including Nuclear Magnetic Resonance (NMR) studies: Kamah A, Cantrelle F X, Huvent I, Giustiniani J, Guillemeau K, Byrne C, Jacquot Y, Landrieu I, Baulieu E E, Smet C, Chambraud B, Lippens G (2016) Isomerization and Oligomerization of Truncated and Mutated Tau Forms by FKBP52 are Independent Processes. J Mol Biol 428, 1080-1090. Indeed, FK1 and FK2 domains of FKBP52 are able to interact with one hexapeptide of Tau (AA 306-311) called PHF6 known to be highly involved in Tau aggregation; Kamah, A, et al. supra, von Bergen M, Friedhoff P, Biernat J, Heberle J, Mandelkow E M, Mandelkow E (2000) Assembly of tau protein into Alzheimer paired helical filaments depends on a local sequence motif ((306)VQIVYK(311)) (SEQ ID NO: 12) forming beta structure. Proc Natl Acad Sci USA 97, 5129-5134.

This hexapeptide, which self-aggregates, is largely used to study amyloid formation; Schirmer C, Lepvrier E, Duchesne L, Decaux O, Thomas D, Delamarche C, Garnier C (2016) Hsp90 directly interacts, in vitro, with amyloid structures and modulates their assembly and disassembly. Biochim Biophys Acta 1860, 2598-2609.

The peptides according to the invention are characterized by their properties as active peptides corresponding to their ability to form a complex with Tau.

### SUMMARY OF THE INVENTION

The invention is broadly directed to peptide fragments of FKBP52 (Gene ID: 2288) that inhibit Tau protein aggregation and in order to ameliorate tauopathies like Alzheimer's Disease (AD). It also involves modifications to these peptides to improve their pharmacokinetic and pharmacodynamic properties. The invention is also broadly directed to prevention or treatment of tauopathies and other diseases, disorders or conditions affected by aggregation of Tau protein. Non-limiting aspects of the invention include the following embodiments. The active peptide according to the invention concerns molecules having the capacity to be bound to Tau protein motifs. Such active peptides can be a fragment of P35 or P50 with or without addition of between 20 residues.

One aspect of the invention is directed to a method for preventing, reducing the severity of, or treating a tauopathy comprising administering to a subject in need thereof a peptide fragment of FK506-binding protein (FKBP52) or a modified form thereof. The tauopathy may be Alzheimer's Disease (AD), familial FTLD-Tau or progressive supranuclear palsy (PSP) or the other tauopathy or tau-related disorders described herein.

In one embodiment, the peptide fragment used in this method comprises, consists essentially of, or consists of P35 (SEQ ID NO: 1). In some embodiments, the peptide fragment comprises, consists essentially of, consists of a modified form of P3S (SEQ ID NO: 1) having backbone protection or a modified N or C terminus or consists of a modified form of P35 (SEQ ID NO: 1) that further comprises a cell penetrating peptide linked to P35 such as YARAAARQARA (SEQ ID NO: 3). As used herein the term “consists essentially of” refers to a peptide that prevents or reduces the severity of a tauopathy or its symptoms or that decreases the incidence or degree of aggregation of Tau protein or of NFTs.

In another embodiment, the peptide fragment used in this method comprises, consists essentially of, or consists of P50 (SEQ ID NO: 2). In some embodiments, the peptide fragment comprises, consists essentially of, consists of a modified form of P50 (SEQ ID NO: 2) having backbone protection or a modified N or C terminus or consists of a modified form of P50 (SEQ ID NO: 2) that further comprises a cell penetrating peptide linked to P50 such as YARAAARQARA (SEQ ID NO: 3). As used herein the term “consists essentially of” refers to a peptide that prevents or reduces the severity of a tauopathy or its symptoms or that decreases the incidence or degree of aggregation of Tau protein or of NFTS.

Another aspect of the invention is directed to a peptide or peptide product such as a modified P35 or P50 peptide comprising a fragment of FKBP52 that inhibits Tau protein aggregation. In some preferred embodiments, the peptide or modified peptide is P35 or P50. The modified peptide many have a modified backbone or a modified C or N terminus. In one embodiment, the modified peptide is CP35 or CP50, which correspond to P35 and P50, that has an N terminal cysteine residue. In some embodiments, the modified peptide may further comprise a cell penetrating peptide, such as the cell penetrating peptide YARAAARQARA (SEQ ID NO: 3).

Another aspect of the invention is the delivering of peptides of the present invention using one or more modalities. The present invention provides vectors that package nucleic acid of the invention encoding peptides. Vectors of the present invention may be used to deliver the nucleic acid or modified nucleic acid encoding a peptide of interest to a cell such as neurons, a local tissue site such as brain or a subject. These vectors may be of any kind, including DNA vectors, RNA vectors, plasmids, viral vectors and particles. Viruses, which are useful as vectors include, but are not limited to lentiviral vectors, adenoviral vectors, adeno-associated viral (AAV) vectors. Vectors can comprise native or non-native promoters operably linked to the nucleic acid coding for peptides of the invention. The promoters selected may be strong, weak, constitutive, inducible, tissue specific, development stage-specific, and/or organism specific.

In some embodiments, the optimal promoter may be selected based on its ability to achieve minimal expression of the peptide of interest of the invention.

In some embodiments, lentiviral vehicles/particles may be used as delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV).

Typically, lentiviral particles making up the gene delivery vehicle are replication defective on their own (also referred to as “self-inactivating”). Lentiviruses are able to infect both dividing and non-dividing cells by its entry through the intact host nuclear envelope (Naldini L et al., Curr. Opin. Biotechnol, 1998, 9:457-463). Lentivirus vectors used may be selected from, but are not limited to pLenti, pLenti6, pULTRA, pInducer20, p197.

Delivery of any nucleic acid coding for peptides of the present invention may be achieved using recombinant adeno-associated viral (AAV) vectors. Such vectors or viral particles may be designed to utilize any of the known serotype capsids or combinations of serotype capsids. Capsids may include but not limited to AAV1, AAV2, AAV3, AAV4, AAV5, AAV6, AAV7, AAV8, AAV9, AAV10, AAV11, and AAV12.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

In some embodiments, nucleic acid coding for the peptide of interest may be administered in one or more AAV particles.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B: Synthetic peptides obtained from FKBP52 inhibit Tau-P301L aggregation in vitro. FIG. 1A: Three dimensional structure of P50 and P35 is generated with Pymol v0.99. FIG. 1B, top panel: ThT fluorescence analysis of Tau-P301L (5 μM, blue/top curve) aggregation (co-incubation with Tau seeds) with or without P35 (50 μM, green/3^{rd} from top curve) or P35 scramble (50 μM, yellow/mid curve) over time (24 hours). Incubation of Tau-P301L without Tau seeds does not show aggregation (grey/bottom curve). Histograms in FIG. 1B, bottom panel represent the fluorescence intensity measured at 24 h. Statistical analysis was performed using Student's t-test; n=3, *p<0.05, ***p<0.001; ±SEM.

FIGS. 2A-2D: DRG neurons treatment with P50-YARA or P35-YARA, FIG. 2A: Images showing the region where the spinal cord was collected from the mouse (red lines/line along mouse back) and the spinal cord sample (top right). The drawing illustrates a cross section of the spinal cord; the cluster of sensory neuron cell bodies (DRGs) situated on the dorsal root of the spinal cord is drawn in blue. FIG. 2B: DRGs dissection from 3 month-old mice. DRG neurons were collected from hTau-P301S mice spinal cords, dissociated and cultured. Synthetic peptides/virus coding for peptides were administrated respectively for 1 week/5 days and maintained during 2 weeks. Soluble and sarkozyl-insoluble fractions of cell lysates were analyzed by western-blot. FIG. 2C: Confocal images showing Rho-P35-YARA (red) in DRG cells (green). White arrows in magnification show the presence of intracellular Rho-P35-YARA. Nucleus is stained by DAPI (blue). FIG. 2D: Western blot analysis of sarkosyl-insoluble fraction obtained from hTauP301S mouse DRG neurons treated or not with YARA-P50 or YARA-P50Sc.

FIGS. 3A-3H. FKBP52 partially colocalizes with Tau in the lysosome. FIG. 3A: Double labeling Immunofluorescence: The FKBP52 signal (green) colocalizes with the Cathepsin D signal (red) of endosomal lysosomal vesicles in neurons of FTLD-Tau patients. Normal lysosomes in healthy neurons (size range: 0.2-0.5 μm) and enlarged lysosomes in AD neurons (size range: 1.5-3 μm) are indicated by arrowheads (see magnification, Scale bar 10 μm). FIG. 3B: Fluorescent levels of neuronal FKBP52 in familial FTLD-Tau neurons quantified by image analysis are significantly decreased compared with neurons in the frontal cortex of age matched controls. Statistical analysis was performed using Student's t-test, n=4; **p<0.01; ±SEM. FIG. 3C: Total frontal cortex homogenates of familial FTLD-Tau patients analyzed by Western-blot and immunoblotted with EC1 (FKBP52) and 6C5 (GAPDH) antibodies. FIG. 3D: FKBP52 quantification in total brain homogenates (FKBP52/GAPDH) in controls and familial FTLD-Tau patients. Student's t-test, n=4; **p<0.01; ±SEM. FIG. 3E: Double labeling of a frontal cortex section from a familial FTLD-Tau patient showing a visibly decreased FKBP52 expression (green, arrow and arrowhead) in neurons with AT180 deposits (red, arrowhead) but not in adjacent neurons without visible AT180 labeling (arrows). FIG. 3F: Scatter diagrams of image analysis results of frontal cortex samples from four familial FTLD-Tau patients showing a significant decrease of FKBP52 in neurons exhibiting AT180 deposits compared to neurons without apparent AT180 deposition. Student's t-test, n=4; ***p<0.001; ±SEM. FIG. 3G: Triple labeling of a single Z-stack plane (0.3 μm) showing the colocalization of the FKBP52 signal (green) with the caspase-cleaved Tau-D421 (TauC3, red) and the lysosomal Cathepsin D (magenta) signals in familial FTLD-Tau brain neurons. FIG. 3H: Fluorescence profiles of FKBP52, TauC3 and Cathepsin D antibodies showing the colocalization of the three signals on a single plane. Scale bar 10 μm.

FIGS. 4A-4E. Decreased expression of FKBP52 in the frontal cortex of PSP patients: FKBP52 decrease in neurons with pathologic Tau deposits is not limited to a specific tauopathy. FIG. 4A: Representative Western-blot analysis of PSP frontal cortex homogenates showing a non-significant decrease of FKBP52 (lower half), probably due to the strong heterogeneity of the neuronal pathology in the frontal cortex of PSP patients. FIG. 4B: Double labeling experiment on the frontal cortex section of a PSP patient. FKBP52 (green) expression in neurons with AT180 deposits (red, arrowheads) is visibly decreased (merge) in comparison to neurons without visible AT180 labeling (arrows). FIG. 4C: Scatter diagrams of cell by cell image analysis of neurons with and without AT180 deposits showing a significantly lower FKBP52 expression in cells exhibiting AT180 NFTs. Statistical analysis was performed using Student's t-test, n=5; **p<0.01; ±SEM. FIG. 4D: Triple labeling experiment on a frontal cortex section from a PSP patient showing FKBP52 (green), TauC3 (red) and Cathepsin D (magenta) colocalization (merge). FIG. 4E: The single Z-stack plane (0.3 μm) fluorescence profiles of a neuron show the superposition of the three signals (arrows on image and fluorescence profiles). Scale bar 10 μm.

FIGS. 5A-5C. Triple labeling experiments showing the lysosomal colocalization of FKBP52 and the early pathologic Tau-pS422 isoform in frontal cortex neurons of patients with different tauopathies. Triple labeling experiments of familial FTLD-Tau (FIG. 5A), AD Braak VI (FIG. 5B) and PSP (FIG. 5C) frontal cortex sections respectively: FKBP52 (green) and Tau-pS422 (red) colocalize (merge) with the endolysosomal marker Cathepsin D in the three tauopathies. Scale bar 10 am. At the lower right of each set of micrographs: superposition of the three respective single plane fluorescence profiles of a single neuron (lower left, scale bar 3 μm).

FIGS. 6A-6F. Image analysis results showing the decrease of FKBP52 expression in neurons with Tau-pS422 deposits in the frontal cortex of patients with different tauopathies. (FIGS. 6A, 6C, 6E): FKBP52 expression (green) is visibly decreased in Tau-pS422 (P422, red) positive neurons of AD Braak VI, familial FTLD-Tau and PSP patients respectively (arrowheads) compared with adjacent neurons devoid of evident Tau-pS422 deposits (arrows). (FIGS. 6B, 6D, 6F): Scatter diagrams of image analysis results showing a significant decrease of FKBP52 expression in Tau-pS422 positive neurons of AD Braak VI, familial FTLD-Tau and PSP patients respectively. Statistical analysis was performed using Student's t-test, *p<0.05, ***p<0.001, ±SEM.

FIGS. 7A-7H. Expression and age variations of FKBP52 and pathologic Tau isoforms in spinal cord neurons of hTau-P301S mice. (FIG. 7A) Representative Western-blot analysis of the spinal cords of 1, 3 and 5 months old hTau-P301S mice showing the progressive accumulation of human Tau and of different pathologic Tau isoforms. NSE was used as a loading control (n=5). (FIG. 7B) Graph illustrating the increase of human Tau and some pathologic Tau isoforms in spinal cords from hTau-P301S mice, beginning at three months and sharply increasing to peak at 5 months of age. (FIG. 7C) Representative Western-blot analysis illustrating the decrease of FKBP52 neuronal expression beginning at three months of age in spinal cords from hTau-P301S mice, compared with control animals. NSE was used as a loading control. (FIG. 7D) FKBP52 quantification in total spinal cord homogenates (FKBP52/NSE) in control and h Tau-P30IS mice. Statistical analysis was performed using Student's t-test, n=5; *p<0.05; ±SEM. (FIG. 7E) Section of the spinal cord of a 5 months-old hTau-P301S mouse double labeled with FKBP52 (green) and AT180 (red) showing the decrease of FKBP52 expression in a neuron with AT180 deposits (arrowheads) compared with adjacent neurons devoid of AT180 (arrows). (FIG. 7F) Scatter diagrams of image analysis results illustrating the significant decrease of FKBP52 in spinal cord neurons from 5 months-old hTau-P301S mice exhibiting AT180 deposits Statistical analysis was performed using Student's t-test, n=5; ***p<0.001; ±SEM. (FIG. 7G) Spinal cord section of a 5 months-old hTau-P301S mouse double labeled with FKBP52 (green) and Tau-pS422 (red) showing the decrease of FKBP52 expression in two neurons exhibiting Tau-pS422 deposits (red, arrowheads) compared with an adjacent neuron devoid of Tau-pS422 deposits (arrows). (FIG. 7H) Scatter diagrams of image analysis results illustrating the significant decrease of FKBP52 in spinal cord neurons from 5 months-old hTau-P301S mice exhibiting Tau-pS422 deposits. Statistical analysis was performed using Student's t-test, n=5; ***p<0.001; ±SEM.

FIGS. 8A-8D. Decrease of neuronal FKBP52 at early asymptomatic stages of neurodegenerative tauopathy in human and mouse. (FIG. 8A) Decrease of the FKBP52 signal (green) in the rare neurons exhibiting Tau-pS422 deposits (red, arrowheads), compared with adjacent neurons devoid of evident Tau-pS422 deposits (arrows), in the frontal cortex of AD Braak IV individuals after double labeling Immunofluorescence. (FIG. 8B) Scatter diagrams of image analysis results of FKBP52 expression in individual neurons from the frontal cortex of AD Braak IV patients after double labeling immunofluorescence illustrating the significant decrease of FKBP52 expression in adjacent neurons with and without Tau-pS422 deposits. Statistical analysis was performed using Student's t-test, n=5; **p<0.01; ±SEM. (FIG. 8C) Decrease of the FKBP52 signal (green) in the rare neurons exhibiting Tau-pS422 deposits (red, arrowheads), compared with adjacent neurons devoid of evident Tau-pS422 deposits (arrows), in the rachis of asymptomatic 1 month-old hTau-P301S mice after triple labeling immunofluorescence. (FIG. 8D) Scatter diagrams of image analysis results of FKBP52 expression in individual neurons from asymptomatic 1 month-old hTau-P301S mice showing the significant decrease of FKBP52 expression in adjacent neurons with and without Tau-pS422 deposits. Statistical analysis was performed using Student's t-test, n=3; *p<0.05; ±SEM.

FIG. 9. Triple labeling experiment on a FTLD-Tau frontal cortex section. FKBP52 (green) colocalizes with pathological Tau-D421 deposits (red) in enlarged cathepsin D positive (magenta) lysosomal vesicles.

FIGS. 10A-10B. Colocalization of FKBP52 and a truncated Tau isoform recognized by the TanC3 antibody with ALP markers. (FIG. 10A) Triple labeling experiment of familial FTLD-Tau frontal cortex showing (above) the colocalization of FKBP52 (green) and the caspase-cleaved Tau-D421 (red) with Rab7 (magenta), a late endosome-lysosome associated small GPTase, and the corresponding immunofluorescence profiles with the colocalization of the three respective signals (below). (FIG. 10B) Triple labeling experiment of a familial FTLD-Tau frontal cortex section showing the colocalization of FKBP52 (green) and the caspase-cleaved TauC3 (red) with the autophagosome marker LC3 (magenta, above) and the associated immunofluorescence profiles showing the colocalization of the three respective signals (below). Scale bar=10 μm.

FIGS. 11A-11D. Decrease of FKBP52 in frontal cortex neurons of CBD and PID patient. (FIGS. 11A, 11B) Triple labeling experiments showing the decrease of FKBP52 (green) in Tau AT180 positive (red) frontal cortex neurons (arrowheads), compared to neurons without apparent AT180 deposition (arrows), of a CBD (A) and a PiD (FIG. 11B) patient respectively. (FIGS. 11C, 11D) Triple labeling experiment showing the colocalization of FKBP52 and TauC3 with the lysosomal marker Cathepsin D in CBD (FIG. 11C) and PiD (FIG. 11D) frontal cortex neurons, and the respective immunofluorescence profiles on a single plane in a neuron with Tau deposits showing the superposition of the three signals. Scale bar=10 μm.

FIGS. 12A-12C. Image analysis results showing the decrease of FKBP52 expression in neurons with Tau deposits in frontal cortex neurons of patients with AD Braak VI. (FIG. 12A): FKBP52 (green) and P62 expression (red) in Tau (Total Tau, magenta) positive neurons of AD Braak VI (arrowheads) compared with adjacent neurons devoid of evident Tau deposits (arrows). (FIG. 12B) Image analysis results show a significant increase of P62 expression in the Tau-positive neurons of AD Braak VI patients. Statistical analysis was performed using Student's t-test, n=4; *p<0.05; ±SEM. (FIG. 12C): Scatter diagram showing P62 labeling intensity levels in single frontal cortex neurons of AD Braak VI patients without and with Tau deposits. Scale bar=10 μm.

FIGS. 13A-13B. Lentiviral (FIG. 13A) or AAV (FIG. 13B) plasmid coding for the peptide P50 and respectively named P197-P50 and AAV-hSyn-P50. MND is a promotor. IRES (internal ribosome entry site) is used to co-express different under the same promotor.

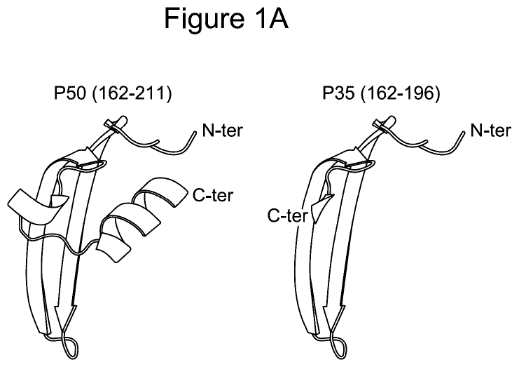

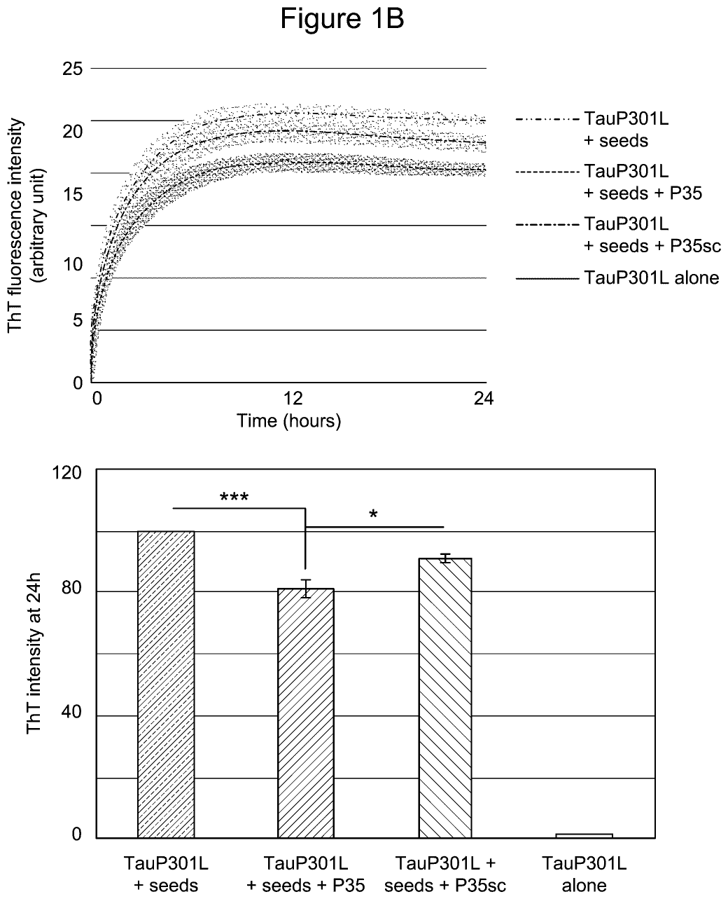

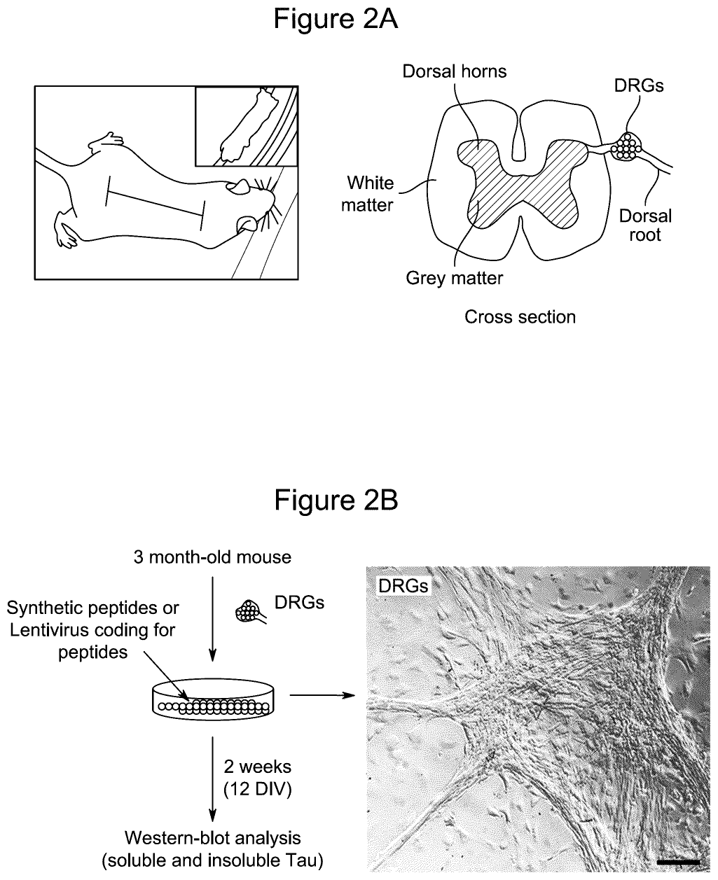

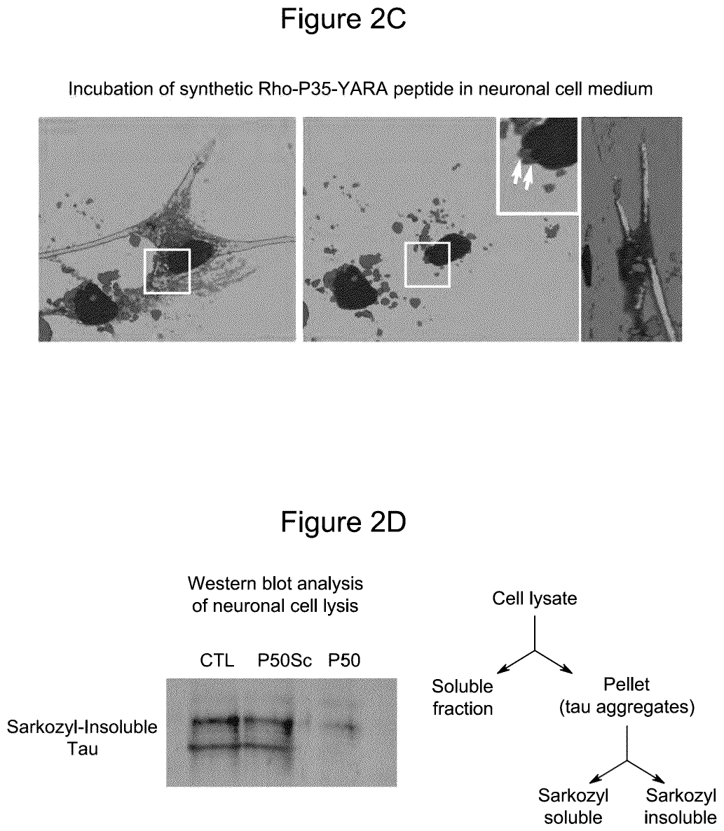

### MATERIALS AND METHODS FIGS. 1 TO 2

Protein purification. Recombinant wild-type Tau and Tan with P301L mutation proteins was expressed in Escherichia coli (E. coli, BL21) and purified as described; Goedert M, Jakes R (1990) Expression of separate isoforms of human tau protein: correlation with the tau pattern in brain and effects on tubulin polymerization. EMBO J 9, 4225-4230. Combs B, Tiernan C T, Hamel C, Kanaan N M (2017) Production of recombinant tau oligomers in vitro. Methods Cell Biol 141, 45-64.

Peptides synthesis
P50
(SEQ ID NO: 2)
(AKPNEGAIVEVALEGYYKDKLFDQRELRFEIGEGENLDLPYGLER
AIQRM),
P50Sc
(SEQ ID NO: 5)
(scramble: AKGNVEAIVEPALEGYDKYKLDFQRELREFIGELGNLED
LYGQPARIERM),
P35
(SEQ ID NO: 1)
(AKPNEGAIVEVALEGYYKDKLFDQRELRFEIGEGE);
P35Sc
(SEQ ID NO: 4)
(scramble: IRFKVLEGLYGEVDEAKEGNEAKAEGLQEYPDFIR);
YARA-P50
(YARAAARQARA(SEQ ID NO: 3)-P50)
YARA-P50Sc
(YARAAARQARA(SEQ ID NO: 3)-P50Sc)
YARA-P35
(YARAAARQARA(SEQ ID NO: 3)-P35)
YARA-P35Sc
(YARAAARQARA(SEQ ID NO: 3)-P35Sc).

Rho-P50-YARA and Rho-P35-YARA were synthesized by “Laboratoire des Biomolécules” (LBM7203, CNRS, École Normale Supérieure, PSL University, Sorbonne Université, 75005 Paris, France) with a purity ≥95% as determined by mass spectrometry.

Effect of P35 on Tau-P30IL aggregation. Tau-P301L protein is used in all aggregation assays. Tau seeds are prepared during a first polymerization: 15UM of monomeric Tau-P301L was incubated at 37° C. in polymerization buffer (PI-buffer): NaH₂PO₄ 25 mM, Na₂HPO₄ 25 mM, NaCl 25 mM, EDTA SmM PH 6,6 with heparin 7.5 μM, Thioflavine-T 50 μM, and Dithiothreitol (DTT) 0.3 mM in a final volume of 100 μl. Fluorescence was monitored every 30 minutes during 25 hours at 480 nm (excitation at 450 nm) using Perkin-Elmer Endvision spectrophotometer. After polymerization, samples (100 μl) were centrifuged at 20,000 g for 1 hour at 4° C. The pellets were resuspended in 100 μl of PI-buffer and sonicated (Branson sonicator) at 40%, 3 times of 1 second, in order to obtain small pre-formed Tau fibrils used as seeds for the second polymerization assay. A volume of 18% of Tau seeds was pre-incubated in the same buffer of the first polymerization, with or without polypeptides, during 1 h at 37° C. Subsequently, 5 μM of monomeric Tau-P301L was added and fluorescence was monitored in the same conditions.

Spin-down assays. After Tau-P301L aggregation for 24 h at 37° C., the samples were collected and centrifuged at 28,000 rpm during 30 min. The supernatant corresponding to the soluble fractions was analyzed by SDS-PAGE. Western-blot was carried out with Tau5 antibody (1:1000; Abcam).

DRG culture and western-blot analyses. DRG neurons were collected from hTau-P301S mice spinal cords as described by Sleigh and dissociated with collagenase (ThermoFisher Scientific, 17100-017) 2900 U/mL during 90 min followed by 0.1% trypsin (Life Technologies, 15090-046) 3 mM EDTA containing DNaseI (50 μg/mL; Sigma-Aldrich, 04536282001) for 5 min; Sleigh J N, Weir G A, Schiavo G, A simple, step-by-step dissection protocol for the rapid isolation of mouse dorsal root ganglia. BMC RES NOTES, 2016, 9, 82.

Cell treatment was stopped using 1×PBS (VWR, X0515-500) supplemented with 2% horse serum (ThermoFisher Scientific, 16050-122), and DRG neurons were dissociated with a Pasteur pipette. Cells were then centrifuged for 5 min at 1,300 g and the pellet was resuspended in DMEM (Fisher Scientific, 12077549) with 9% fetal bovine serum. After filtration on a 0.40 μm filter followed by centrifugation 5 min at 1,300 g. the pellet was resuspended in neurobasal medium complemented with B27 (50×), L-glutamine (100×) (Life Technologies, 11530536 and 25030024 respectively), NGF 2.5 S (27 ng/ml; ThermoFisher, 13257-019) and distributed in 2 wells of a 6-well culture plate. Cell cultures were maintained for 15 days in vitro (DIV) and the cell medium complemented with mitomycine C (Sigma-Aldrich, M4287) and 5-Fluorodeoxy-uridine (Sigma-Aldrich, F0503) was changed twice a week. Cells were harvested by scraping, centrifuged for 10 min at 200 g and the pellet was homogenized using PotterElvehjem 18 in cold extraction buffer (50 mM Tris-HCl, pH 7.5, 100 mM NaCl, 0.5% SDS, 2 mM CaCl2, 2 mM MgCl2) supplemented with a cocktail of protease and phosphatase inhibitors (Roche, 11836145001). Cell lysates were then centrifuged 10 min at 17,000 g, the supernatant (soluble fraction) was kept and the pellet was homogenized using PotterElvehjem 18 in cold extraction buffer supplemented with 1% sarkosyl (Sigma-Aldrich, 19150). Lysates were then centrifuged in a polycarbonate tube (Beckman, 343775) at 100,000 g during 40 min at 4° C. The pellet (sarkozyl insoluble fraction) was boiled in Laemmli sample buffer for 10 min. Each sample was analyzed on a 10% SDS Page gel and transferred onto iBlot™ Gel Transfer membranes (Invitrogen, IB23001, IB24002). Following transfer, membranes were incubated overnight with the appropriate antibodies. Species specific, peroxidase conjugated, secondary antibodies were subsequently used to perform enhanced chemiluminescence (ThermoFisher Scientific, 32106). Images were recorded with the GeneGnome5 (Syngene). Quantification was performed using Genetools analysis software (Syngene).

DRGs treatment with peptides. DRG cells, collected from a hTau-P30IS mice spinal cord, were dissociated and grown on a 6-well tissue-culture plate for 3 days. After changing cell medium, DRG were treated or not with YARA-P50 or YARA-P35 peptides at 200 nM three times per week during two weeks. Cell medium incubation of YARA-P50Sc or YARA-P35Sc at the same concentration was used as control. Cells were then washed with PBS Ix, detached and centrifuged at 1,200 g for 5 min. After cell lysis, lysates were then analyzed by western-blot as described in “DRG culture and western-blot analyses”.

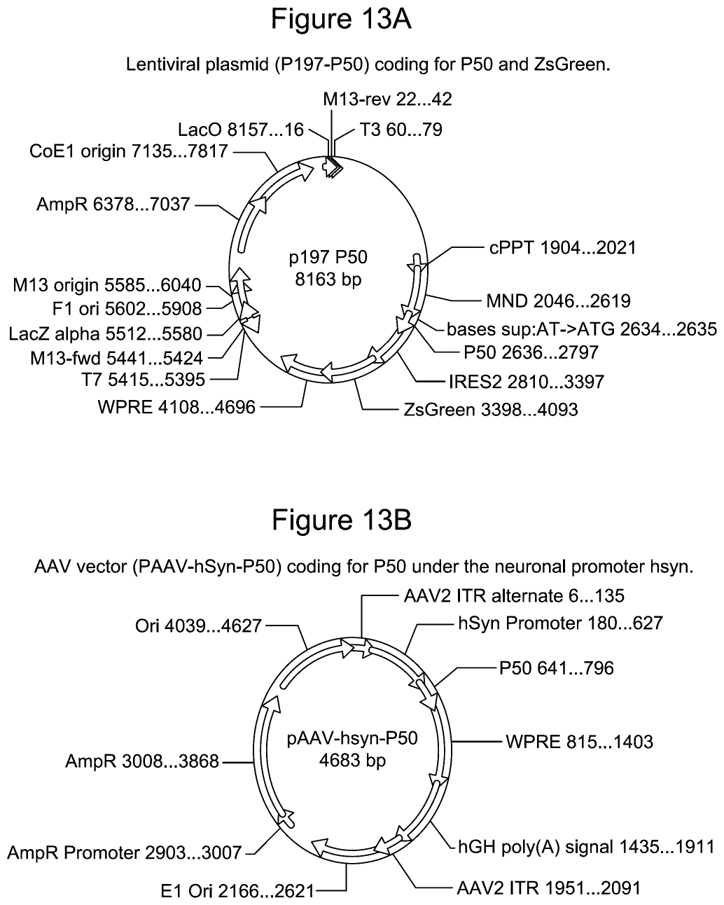

Lentiviral cell transduction. In case of the selected peptides are unmodified in their amino acid sequences, we have also the possibility to express them by cellular internalization of a lentivirus vector engineered to produce specific cDNA encoding proteins or peptides of interest and simultaneously a non-chimeric ZsGreen fluorescent protein (FIG. 13A). The simultaneous detection of ZsGreen proteins will help us to detect transduced neurons that express peptides of interest. Those vectors have been already designed and are currently available. The cDNA encoding P50/P35 was inserted into the restriction site of the 197 plasmid (pRRLsin-MND-MCS-Ires2-ZsGreenWPRE, Vectorology platform Vect′UB, U1035 Inserm, Bordeaux) to obtain the concomitant expression or P50/P35 and ZsGreen proteins. Lentiviruses containing the cDNA coding for FKBP52 derived-peptides were generated at the vectorology platform Vect′UB (U1035 Inserm, Bordeaux). In case of the selected peptides are unmodified in their amino acid sequences, we propose to directly express them by cellular internalization of an AAV vector engineered to produce specific cDNA encoding for each peptides under the control of a neuronal promoter (FIG. 16 B). DNA sequence coding for P50 or P35 was sub-cloned to an AAV vector obtained from Addgene (N°50465, AAV-eGFP) where eGFP gene has been removed and replaced by peptides expressing gene (AAV-peptide such as AAV-P50 or AAV-P35).

Confocal microscopy analyses. DRG neurons from one adult mouse were distributed in 6 wells of a 12 well culture plates. Cells were treated with 200 nm of fluorescent peptides (Rho-peptide-YARA) three times per week during two weeks. Cells incubation were washed two times with 1×PBS (VWR, X0515-500) for 1 min and then fixed with 4% PFA (Merck, 104005) for 10 min at room temperature. After blocking with Sea Block blocking buffer (SBBB; 1:10; ThermoFisher Scientific, 37527) in 1×PBS for 1 h at room temperature, cells were incubated with the primary antibody anti-TUBB3/βIII tubulin (1:2000, Abcam, ab18207) diluted in 1×PBS SBBB (1:10), overnight at 4° C., followed by staining with Alexa Fluor 488-conjugated secondary antibody (Life Technologies; 1:2000) for 45 min at 37° C. Nuclei were stained with DAPL. The coverslips examined by epifluorescence under a Leica SP8 confocal microscope. Images were processed using ImageJ FIJI, 3D Viewer software.

### Example 1: Results and Discussion FIG. 1

Synthetic peptides obtained from FKBP52 inhibit Tau-P301L aggregation in vitro. As shown FIG. 1, we have identified a peptide issued from FKBP52 of 35 AA called P35 that is able to prevent Tau-P301L assembly (FIGS. 1A and B). Using Thioflavine T fluorescence assays, we observed that Tau-P30IL aggregate formation is decreased when incubated in the presence of P35 (80.6%, +/−2.8%, sem) in comparison to P35 scramble (P35sc, 91.4% (+/−1.5%, sem) used as control (FIG. 1 B, green and yellow curves/histograms; t-test, *p<0.05). Using the same approach, in vitro incubation of Tan-P301L with P50 showed comparable effects than P35 (data not shown). The shorter P35 peptide might present useful proprieties, such as a stronger bio-availability, an easier and reproducible synthesis or possible fewer side effects, and then must be studied for these reasons, along with P50, in its ability to inhibit Tau aggregation in cellular and animal models of tauopathies.

### Example 2: Results and Discussion FIG. 2

Tauopathic DRG (Dorsal Root Ganglion) neuronal cells treatment with YARA-PSO/YARA-P35 tends to show a decrease of Tau-P301S aggregation. In order to test our selected peptides in their ability to inhibit Tau aggregation in cellulo, we treated by two different ways (viral infection or synthetic peptide administration) primary cultures of DRGs (Dorsal Root Ganglions) neurons obtained from the hTau-P301S transgenic mouse model. DRGs are clusters of sensory neuron cell bodies dissected from the dorsal roots of the spinal cord of adult hTau-P301S mice which develop Tau aggregates over time similarly to those found in human tauopathies (FIGS. 2A and B; Allen B, Ingram E, Takao M, Smith M J, Jakes R, Virdee K, Yoshida H, Holzer M, Craxton M, Emson P C, Atzori C, Migheli A, Crowther R A, Ghetti B, Spillantini M G, Goedert M (2002) Abundant tau filaments and nonapoptotic neurodegeneration in transgenic mice expressing human P301S tau protein. J Neurosci 22, 9340-9351). To allow PSO and/or P35 to directly penetrate through the bio-membranes into neuronal cells, we covalently conjugated a cell-penetrating peptide called YARA (Ac-YARAAARQARA-NH2) (SEQ ID NO: 3), derived from human immunodeficiency virus-1 trans-activator of transcription with P50 and P35; Guidotti G, Brambilla L, Rossi D (2017) Cell-Penetrating Peptides: From Basic Research to Clinics. Trends Pharmacol Sci 38, 406-424.

To investigate whether YARA-conjugated peptides were able to penetrate cultured DRG neurons, we combined those peptides to the fluorescent rhodamine (red fluorescence) and analyzed neuronal cells by confocal microscopy (FIG. 2C). As shown in the figure by white arrows, we detected the presence of red fluorescent peptides inside neurons (green fluorescence) showing that Rho-P35-YARA peptides can penetrate neuronal cells in our experimental conditions. Same results were obtained with Rho-P50-YARA peptides (data not shown). We then evaluated the impact of YARA-P35 or YARA-P50 peptides on Tau aggregation after their cell medium incubation for 15 days. Western blot analyses tended to show that YARA-P50 incubation in DRG cells medium for 15 days generated a decrease of insoluble Tau forms when compared to YARA-P50Sc used as control (FIG. 2D).

### Example 3: Peptide Synthesis for Fragments of FKBP52

This example discloses or details additional procedures and or steps for synthesizing the FKBP52 derived peptides described herein. The general synthesis of fragments of FKBP52 was carried out using two distinct techniques—Boc chemistry and Fmoc chemistry. Boc chemistry was used for smaller fragments—up to 35 residues (amino acids) in length. Longer fragments required the use of Fmoc chemistry on a microwave synthesizer with peptide backbone protection.

In addition to the synthesis of the fragments of the protein, a strategy of attachment of the peptide fragments to peptide vectors (or cell penetrating peptides (CPPs)) was undertaken. These vectors can transport the P35 and P50 peptide “cargos” across cell membranes and enhance their activity. In each case the CPP was attached to the peptide fragments using a disulfide bond. In a typical reducing cellular environment, the formed disulfide peptide bond is broken, and the CP35 and CP50 peptides are released. This strategy required the addition of a cysteine residue to the N-terminus of all peptides. As such, fragments are named by their length (P35 is a 35 membered peptide from FKBP52, P50 is a 50 membered peptide) and the equivalent peptide with a Cysteine on the N-terminus (CP35—is the P35 peptide with a cysteine, CP50—is the P50 peptide with a cysteine). Selective formation of the CPP-P35 and CPP-P50 peptides was carried out using Cys(Npys) attachment to the CPPs. This allows for preferential disulfide bond formation as the free cysteine of the CP35 and CP50 will preferentially attach to the Cys (Npys) and not another CP35 or CP50.

### Peptide Synthesis Technique 1:

Boc chemistry for P35, CP35, and cell penetrating peptides (CPPs).

P35: and CP35
P35:
(SEQ ID NO: 1)
Ac-AKPNEGAIVEVALEGYYKDKLFDQRELRFEIGEGE-
NH₂. (STFA).
CP35:
(SEQ ID NO: 7)
Ac-CAKPNEGAIVEVALEGYYKDKLFDQRELRFEIGEGE-
NH₂. (STFA).

P35 and CP35 were synthesized using Boc solid phase peptide synthesis with in situ neutralization (M Schnölzer el. al. (Int J Pept Protein Res. 1992 September-October; 40(3-4):180-93. doi: 10.1111/j.1399 3011.1992.tb00291.x.)

This chemistry used is HBTU in DMF mediated couplings between an activated amino acid in the presence of the resin bound amine.tfa salt of the growing peptide. Amino acids were added sequentially, with a standard sequence of 1) coupling of the amino acid, 2) washing of the peptide resin, 3) deprotection of the introduced amino acid, and 4) washing. Finished peptides on were cleaved, deprotected and purified as outlined below.

Protocol: Dry MBHA resin (172 mg, 0.1 mmol, loading 0.58 mmol/g) was swollen in DCM in a fritted syringe for five minutes and the solvent removed by filtration. The resin was swollen for a second time for ten minutes in DCM and the solvent removed. The resin was then washed three times in fresh DMF, for one minute each time, with filtering of the solvent after incubation. Coupling of the first amino acid was carried out with the corresponding Boc protected amino acid using five equivalents of Boc protected amino acid, four point nine equivalents of HBTU and ten equivalents of DIPEA in DMF. Coupling was carried out for one hour. The resin was then washed four times in DMF. Boc deprotection was then carried out by incubation with TFA twice; once for thirty seconds and once for one minute. The TFA was removed by filtration and the resin washed four times in DMF. Coupling of the subsequent Boc-protected amino acid was carried out directly without pre-neutralization of the amine. TFA salt on the resin. Each subsequent amino acid (5 equivalents) was activated with DIPEA (20 equivalents) in the presence of HBTU (4.9 equivalents) added to the resin and coupled for 1 hour.

P35: The completed peptide was acetylated with Ac₂O (20 equivalents) and DIPEA (5 equivalents) in DMF for 5 minutes. The peptide-resin was washed with DMF four times, DCM three times and MeOH three times before being dried under a vacuum.

CP35: Addition of the Cys (Mob) residue was accomplished using Boc-Cys (Mob)-OH (5 equivalents) HBTU (4.9 equivalent) and DIPEA (10 equivalents) for one hour. The Boc group was deprotected using TFA for 30 seconds, followed by one minute. The completed peptide was acetylated with Ac₂O (20 equivalents) and DIPEA (S equivalents) in DMF for 5 minutes. The peptide-resin was washed with DMF four times, DCM three times and MeOH three times before being dried under a vacuum.

Cleavage of the P35 and CP35 peptides with concomitant deprotection of the protecting groups (excepting Cys (Npys)) was carried out using hydrofluoric acid (˜ 1 mL/g)/DMS (250 μL/g)/Anisole (750 μL/g). The cleavage solution was removed in vacuo and the peptide-resin cocktail was precipitated in ice-cold diethyl ether. The precipitate was filtered to remove the ether. The peptide resin residue was dissolved in TFA, filtered from the resin and dried under vacuum. The crude product was freeze-dried from acetonitrile/water. The crude products were purified using RP-HPLC on a C-18 column using a gradient of acetonitrile in water. The peptides were identified by MALDI-TOF spectrometry.

### Activated CPPs (Cell Penetrating Peptides) for Conjugation:

Arg₉:
(SEQ ID NO: 10)
Ac-C(Npys)RRRRRRRRR-NH₂. (9TFA)
YARA:
(SEQ ID NO: 11)
Ac-C(Npys)YARAAARQARA-NH₂. (3TFA).

Cell penetrating peptides were synthesized using the same Boc chemistry as above on a 0.1 mmol scale and using the same MBHA resin. Cell penetrating peptides contain an activated Cys(Npys) residue which undergoes selective disulfide formation with a free cysteine under controlled conditions. The Cys (Npys) was added to the N-terminus of the cell penetrating peptide using Boc-Cys (Npys)-OH (3 equivalents) and DCC (3 equivalents) in DCM for 1 h. The Boc group was removed by TFA, 1×30 seconds and 1×1 minute. The completed peptide was acetylated with Ac₂O (20 equivalents) and DIPEA (5 equivalents) in DMF for 5 minutes.

Cleavage of the cell penetrating peptides with concomitant deprotection of the protecting groups (excepting Cys (Npys)) was carried out using hydrofluoric acid (˜1 mL/g) and anisole (750 μL/g). The cleavage solution was removed in vacuo and the peptide-resin cocktail was precipitated in ice-cold diethyl ether. The precipitate was filtered to remove the ether. The peptide resin residue was dissolved in TFA, filtered from the resin and dried under vacuum. The crude product was freeze-dried from acetonitrile/water. The crude products were purified using RP-HPLC on a C-18 column using a gradient of acetonitrile in water. The peptides were identified by MALDI-TOF spectrometry.

### Peptide Synthesis Technique 2

Microwave Fmoc chemistry with backbone protection for the P50 and CP50 peptides.

P50:
(SEQ ID NO: 2)
AcAKPNEGAIVEVALEGYYKDKLFDQRELRFEIGEGENLDLPYGLERAI
QRM-NH₂. (7TFA)
CP50:
(SEQ ID NO: 6)
Ac-CAKPNEGAIVEVALEGYYKDKLFDQRELRFEIGEGENLDLPYGLER
AIQRM-NH₂. (7TFA)

CP50 Synthesis of the P50 peptide required the use of a microwave, backbone protection, and a low loading PEG matrix resin. Standard Fmoc synthesis of P50 was unsuccessful with coupling failing after about 15 amino acid residues. At this point only deletion sequences were detected (peptide sequences missing one or more amino acids) by MALDI-TOF. Failure was most likely due to aggregation of the growing peptide chain. To combat this problem, a low loading resin matrix was used with microwave heating. Heating decreases aggregation by unfolding the peptide chain and liberating the N-terminus for coupling with an activated amino acid. The use of backbone deprotection (with Fmoc-Gly (DMB) residues blocks H-bonding and hence aggregation and facilitates coupling with incoming activated amino acids.
P50 with Backbone Protection:

(SEQ ID NO: 2)
Ac-AKPNEGAIVEVALEGYYKDKLFDQRELRFEIGEGENLDLPYGLERA
IQRM-NH₂

Protocol: The P50 peptide was carried out using Fmoc solid phase peptide chemistry on an automated CEM liberty microwave synthesizer. Synthesis of the peptide required both microwave heating and protection of the peptide backbone via the incorporation of Fmoc-Gly(DMB) residues in order to achieve successful formation of the product. The peptide was synthesized on a 0.1 mmol scale on a low loading resin chemMatrix resin (0.22 mmol/g) with inclusion of Gly(DMB) for the first, third and fourth glycine residues (marked in red). Sequential coupling of a five-fold excess of amino acids was carried out using premade solutions of amino acids in DMF (0.2 M). All residues excepting Fmoc-Arg(Pbf) were double coupled (coupled twice before deprotection) with DIC (1 M in DMF) Oxyma (1 M in DMF) initially for 15 seconds at 75° C., then 90° C. for 110 seconds. Fmoc-Arg(Pbf)-OH coupling was carried out for 1500 seconds at 25° C. and then 75° C. for 120 seconds. Fmoc deprotection of the resin and each amino acid was carried out using a solution of 4-methyl piperidine in DMF for 15 seconds at 75° C., then 110 seconds at 90° C. Fmoc deprotection of Fmoc-Arg (Pbf)-OH was carried out for 210 seconds at 75° C. The completed peptide was acetylated with Ac₂O (20 equivalents) and DIPEA (5 equivalents). The peptide-resin was washed with DMF four times, DCM three times and MeOH three times before being dried under a vacuum. Cleavage of the peptide with concomitant deprotection of the protecting groups was carried out using a cocktail of degassed TFA/TIS/H₂O (90:5:5, 7 mL/g resin) for three hours. The cleavage cocktail was removed under vacuum and the crude peptide precipitated in ice cold degassed diethyl ether. The precipitate was centrifuged three times with fresh ether at 5000 RPM in a 15 or 50 ml Falcon tube. The crude product was purified using RP-HPLC on a C-18 column using a gradient of acetonitrile in water. The peptide was identified by MALDI-TOF spectrometry.

Conjugation of peptides to Cell penetrating peptides: Conjugation of CP35 and CP50 with cell penetrating peptides was carried out in a mixture of acetonitrile/10 mM ammonium acetate. To CP35 (2 mg, 1 equivalent) under an atmosphere of Argon was added degassed acetonitrile (300 μL), was added 10 mM ammonium acetate (700 μL). To this solution was slowly added C(Npys)YARA (1 equivalent) in degassed 10 mM ammonium acetate (1 mL). The solution immediately turned bright yellow. The solution was mixed for 5 minutes and purified by RP-HPLC on a C18 column with an acetonitrile gradient. The eluted purified peptide was freeze-dried and identified by MALDI-TOF mass spectrometry.

Amino acids (single and 3-letter codes) and protecting
groups for Boc Solid Phase Peptide Synthesis:

| | | | |
|---|---|---|---|
| A | Ala | Alanine | Boc-Alanine-OH |
| C | Cys | Cysteine (for CPPs) | Boc-Cys(Npys)-OH |
| C | Cys | Cysteine (for P35 and P50) | Boc-Cys(Mob)-OH |
| D | Asp | Aspartic acid | Boc-Asp(Bzl)-OH |
| E | Glu | Glutamic acid | Boc-Glu(Bzl)-OH |
| F | Phe | Phenyalanine | Boc-Phe-OH |
| G | Gly | Glycine | Boc-Gly-OH, |
| I | Ile | Isoleucine | Boc-Ile-OH |
| K | Lys | Lysine | Boc-Lys(2-Cl-Z)-OH |
| L | Leu | Leucine | Boc-Leu-OH |
| M | Met | Methionine | Boc-Met-OH |
| N | Asn | Asparagine | Boc-Asn(Xan)-OH |
| P | Pro | Proline | Boc-Pro-OH |
| Q | Gln | Glutamine | Boc-Gln-OH |
| R | Arg | Arginine | Boc-Arg(Tos)-OH |
| V | Val | Valine | Boc-Val-OH |
| Y | Tyr | Tyrosine | Boc-Tyr(2-Br-Z)-OH |

Amino acid (single and 3-letter codes) and protecting
groups for Fmoc Solid Phase Peptide Synthesis:

| | | | |
|---|---|---|---|
| A | Ala | Alanine | Fmoc-Ala-OH |
| C | Cys | Cysteine | Fmoc-Cys(Trt)-OH |
| D | Asp | Aspartic acid | Fmoc-Asp(tBu)-OH |
| E | Glu | Glutamic acid | Fmoc-Glu(tBu)-OH |
| F | Phe | Phenyalanine | Fmoc-Phe-OH |
| G | Gly | Glycine | Fmoc-Gly-OH, Fmoc-Gly(DMB)-OH |
| I | Ile | Isoleucine | Fmoc-Ile-OH |
| K | Lys | Lysine | Fmoc-Lys(Boc)-OH |
| L | Leu | Leucine | Fmoc-Leu-OH |
| M | Met | Methionine | Fmoc-Met-OH |
| N | Asn | Asparagine | Fmoc-Asn(Trt)-OH |
| P | Pro | Proline | Fmoc-Pro-OH |
| Q | Gln | Glutamine | Fmoc-Gin(Trt)-OH |
| R | Arg | Arginine | Fmoc-Arg(Pbf)-OH |
| V | Val | Valine | Fmoc-Val-OH |
| Y | Tyr | Tyrosine | Fmoc-Tyr(tBu)-OH |

Abbreviations:

| | |
|---|---|
| 2-Cl-Z | 2-chlorobenzyloxycarbonyl |
| 2-Br-Z | 2-bromobenzyloxycarbonyl |
| Ac | Acetyl |
| Ac₂O | Acetic anhydride |
| Boc | tert-butyloxycarbonyl |
| Bzl | Benzyl |
| DCC | Dicyclohexylcarbodiimide |
| DCM | Dichloromethane |
| DIC | Diisopropylcarbodiimide |
| DIPEA | N,N-Diisoproylethylamine |
| DMF | Dimethylformamide |
| DMS | Dimethylsulfide |
| Fmoc | Fluorenylmethoxycarbonyl |
| HBTU | N,N,N′,N′-Tetramethyl-O-(1H-benzotriazol-1-yl)uronium hexafluorophosphate |
| MALDI-TOF | Matrix Assisted Laser Desorption Ionisation Time Of Flight mass spectrometry |
| MeOH | Methanol |
| Mob | 4-Methoxybenzyl |
| Npys | 3-nitro-2-pyridylthio |
| Oxyma | Ethyl cyano(hydroxyimino)acetate |
| Pbf | 2,2,4,6,7-pentamethyldihydrobenzofuran-5-sulfonyl |
| RP-HPLC | Reversed phase high pressure liquid chromatography |
| tBu | tert-butyl |
| TIS | Triisopropylsilane |
| TFA | Trifluoroacetic acid |
| •TFA | Trifluoroacetate salt |
| Tos | Tosyl |
| Trt | Trityl |
| Xan | Xanthyl |

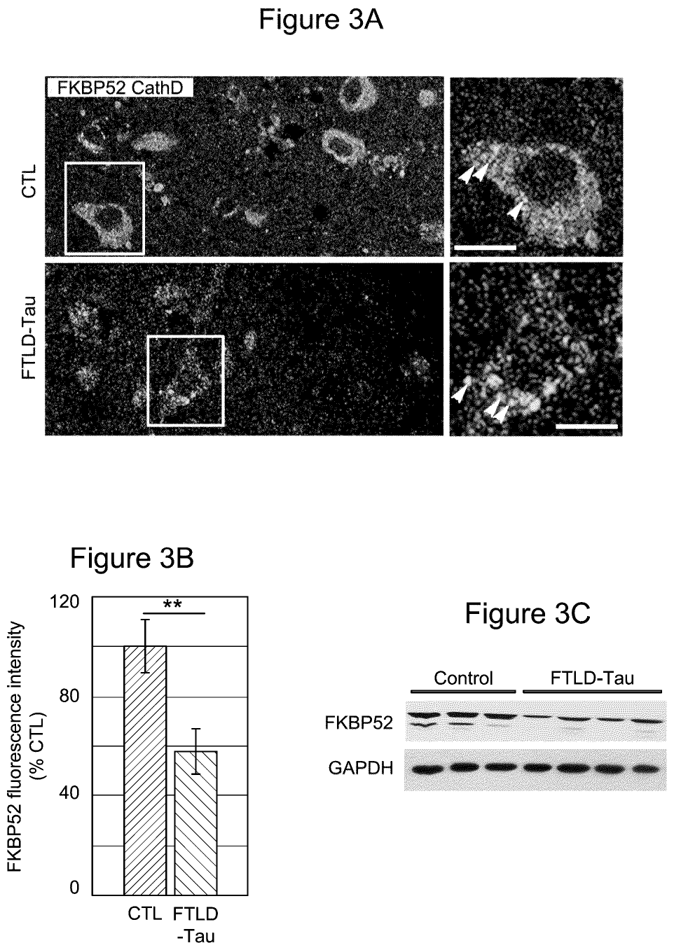

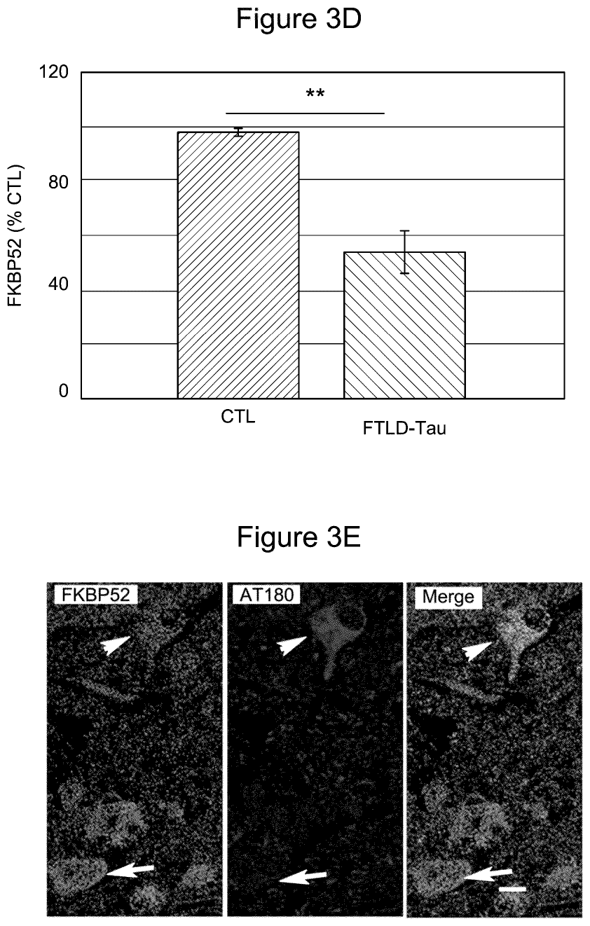

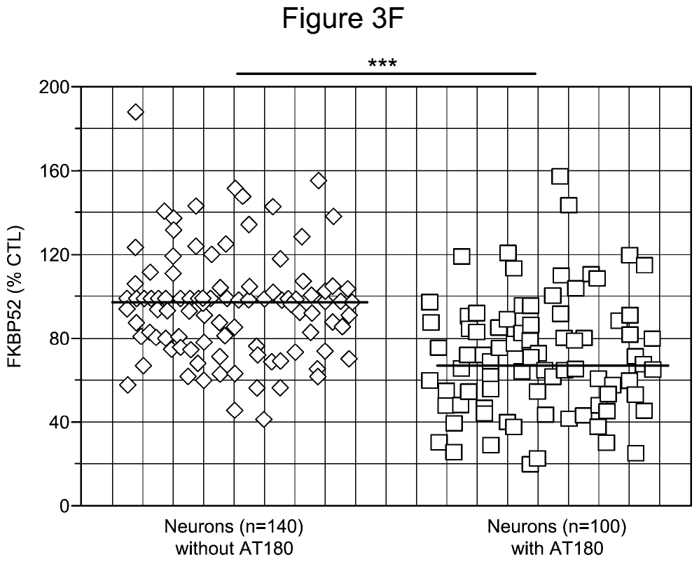

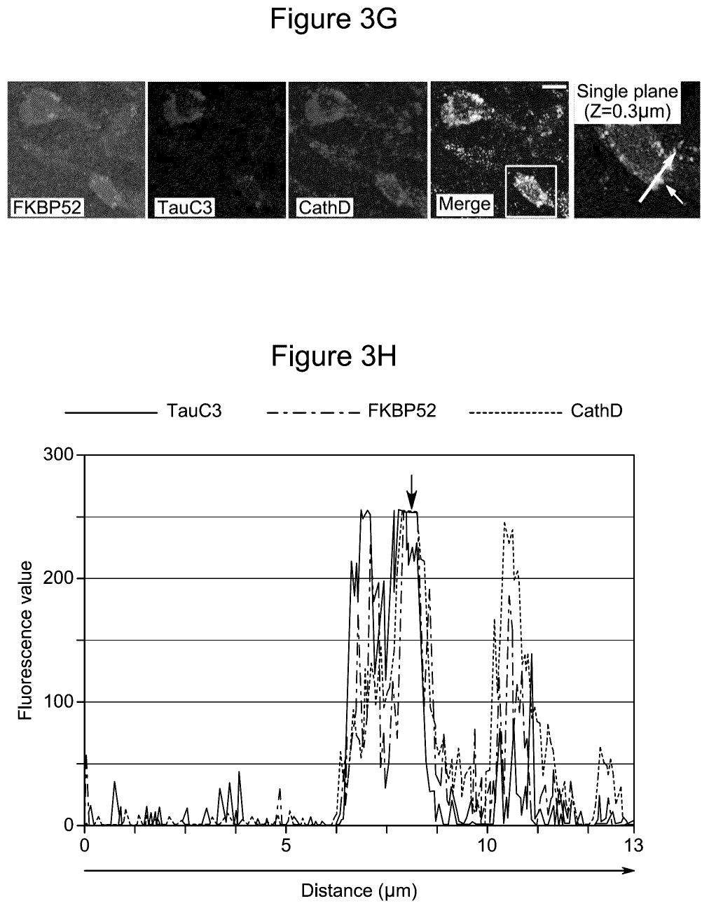

### Example 4: Figure from Meduri et al (FIGS. 3 to 12)

Concomitant neuronal Tau deposition and FKBP52 decrease is an early feature of different human and experimental tauopathies. Using cell by cell immunohistofluorescence analyses and quantification of FKBP52 on postmortem brain samples of some human tauopathies and on hTau-P30IS mice spinal cords, the inventors described FKBP52 decrease and its localization with early pathological Tau forms in the neuronal autophagy-lysosomal pathway in various tauopathies and hTau-P301S mice, see Meduri, Geri et al. ‘Concomitant Neuronal Tau Deposition and FKBP52 Decrease Is an Early Feature of Different Human and Experimental Tauopathies’. J. ALZHEIMER'S DISEASE, 1 Jan. 2023: 313-331, incorporated by reference for all purposes.

As shown, FKBP52 decreases early during the pathologic process as it occurs in rare neurons with Tau deposits in the marginally affected frontal cortex region of AD Braak IV brains and in the spinal cord of symptomless 1-month-old hTau-P301S mice. FKBP52 plays a significant role in cellular signaling and could also be involved in Tau clearance. The data provided in the Example further support the therapeutic efficacy of the methods disclosed herein. The administration of the peptides such as P35 and P50 peptides disclosed herein provides a foundation for prevention of FKBP52 decrease or the restoration of its normal expression at early pathologic stages, in tauopathies including AD, familial FTLD-Tau and PSP.

The inventors show herein that neuronal FKBP52 decrease and localization with truncated-Tau-D421 in the ALP are not limited to AD but are also detected in familial FTLD-Tau and PSP brains and are strongly linked with Tau pathogenesis, suggesting a common mechanism for the abnormal FKBP52 depletion in different tauopathies. They also find by IHF analysis of pathologic human brains that FKBP52 decrease is not restricted to Tau-D421-bearing neurons, which might be apoptotic, but is also detected in Tau-pS422 immunoreactive cells. Tau cleavage by caspases at D421 is known to be inhibited by serine 422 phosphorylation (Tau-pS422), suggesting that the decrease of FKBP52 they observe is not necessarily related to Tau-D421 accumulation but might happen at an earlier pathological stage. Analysis of spinal cord neurons from transgenic hTau-P30IS mice also shows that FKBP52 decrease happens concomitantly with pathological Tau accumulation and confirms that this phenomenon is not restricted to caspase-cleaved Tau-D421 accumulation. Finally, using cell by cell IHF analysis, the inventors found a significant FKBP52 depletion in the rare neurons with Tau-pS422 deposits present in scarcely affected frontal brain regions of AD Braak IV patients and in spinal cords of symptomless young hTau-P301S mice. These affected neurons are detected at an early stage of the pathologic process and in mostly spared areas that will nevertheless be strongly impacted later on with disease progression. This suggests that the FKBP52 decrease in Tau-pS422 positive neurons in these hardly affected regions is an early pathologic event.

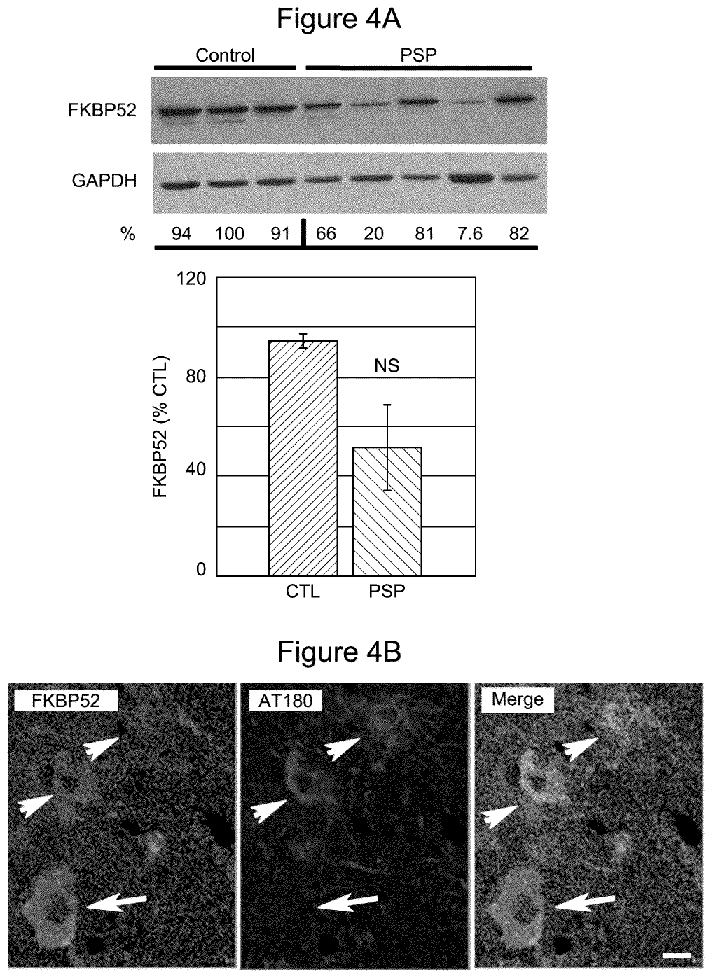

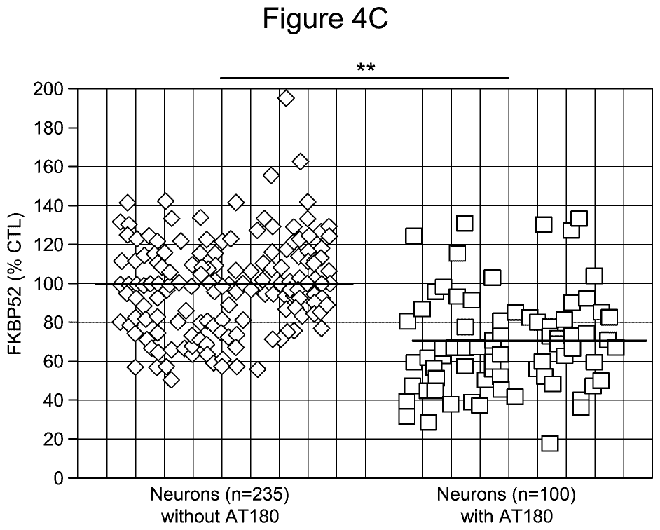

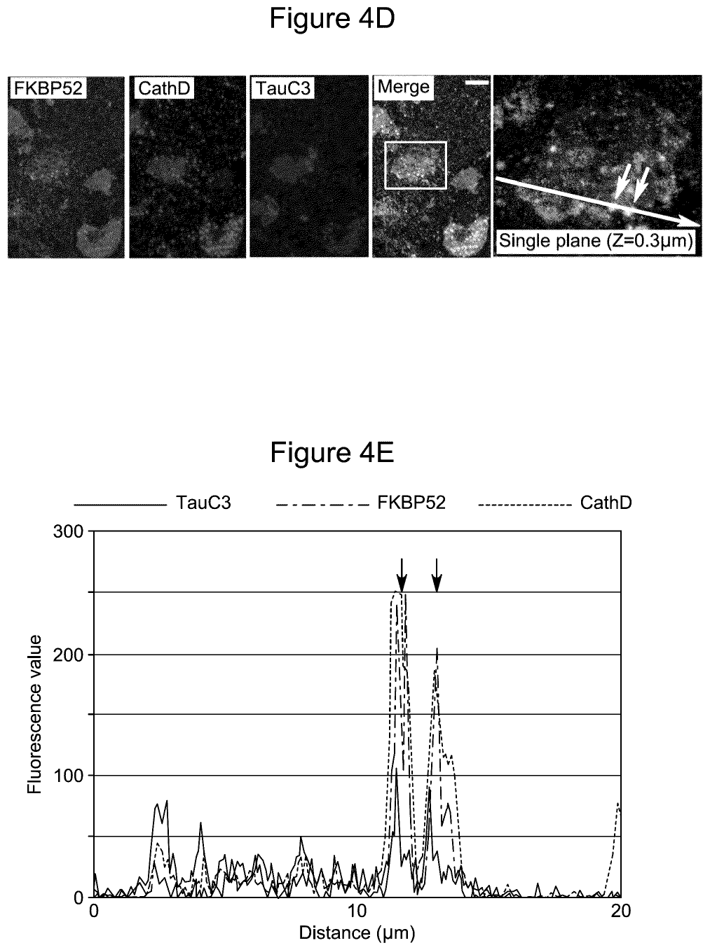

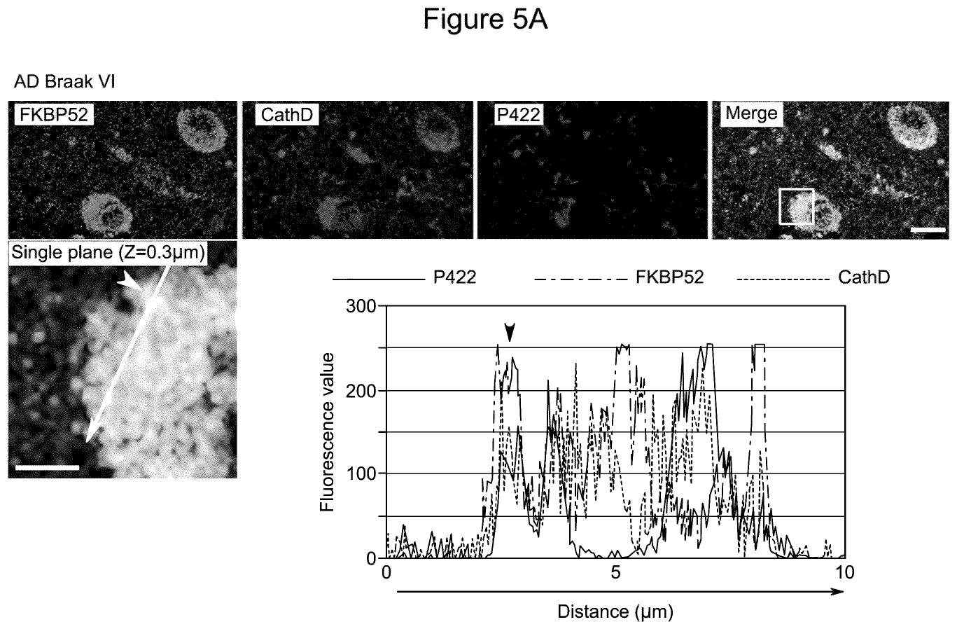

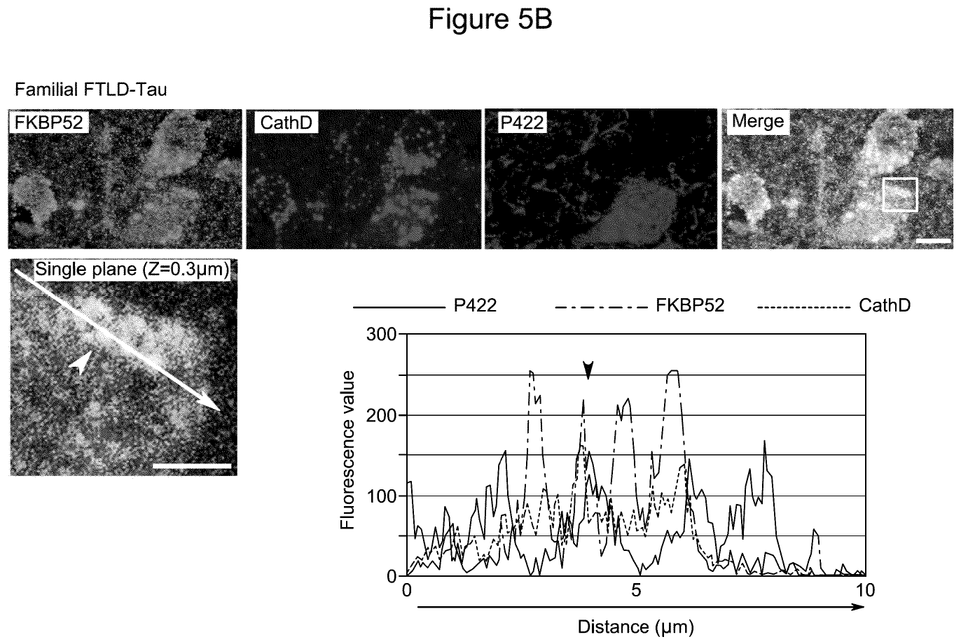

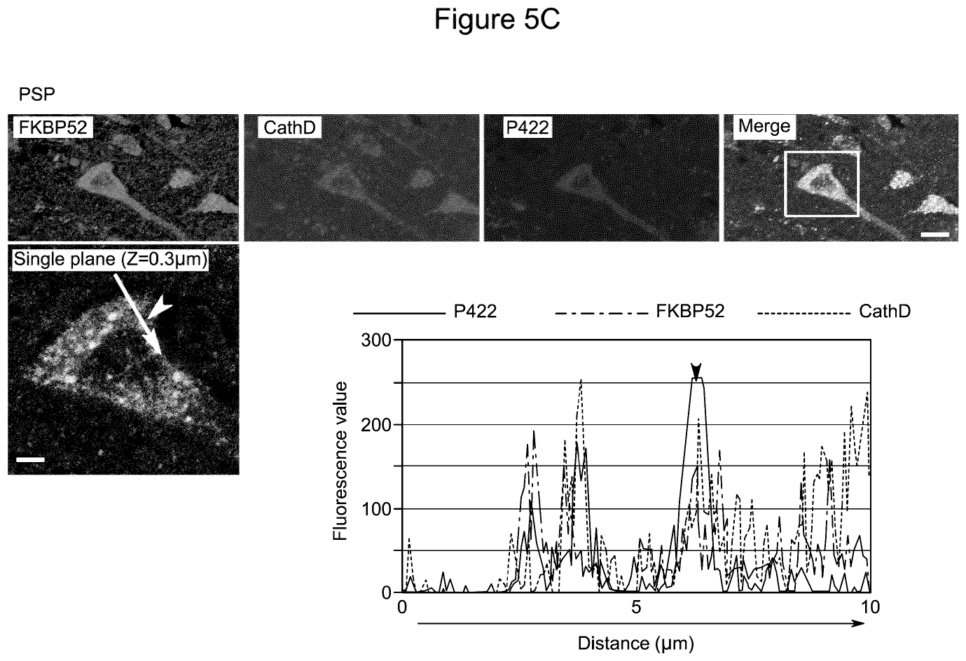

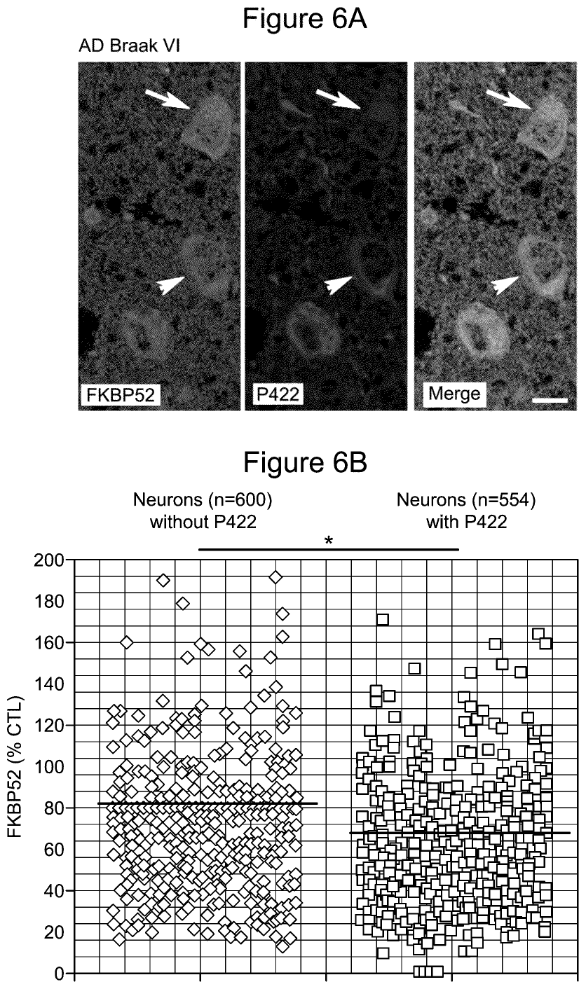

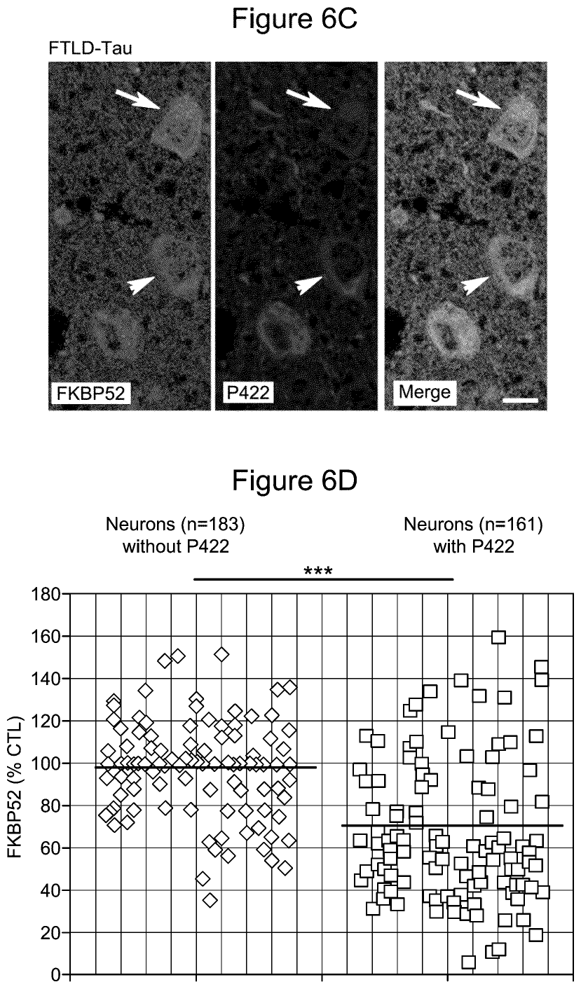

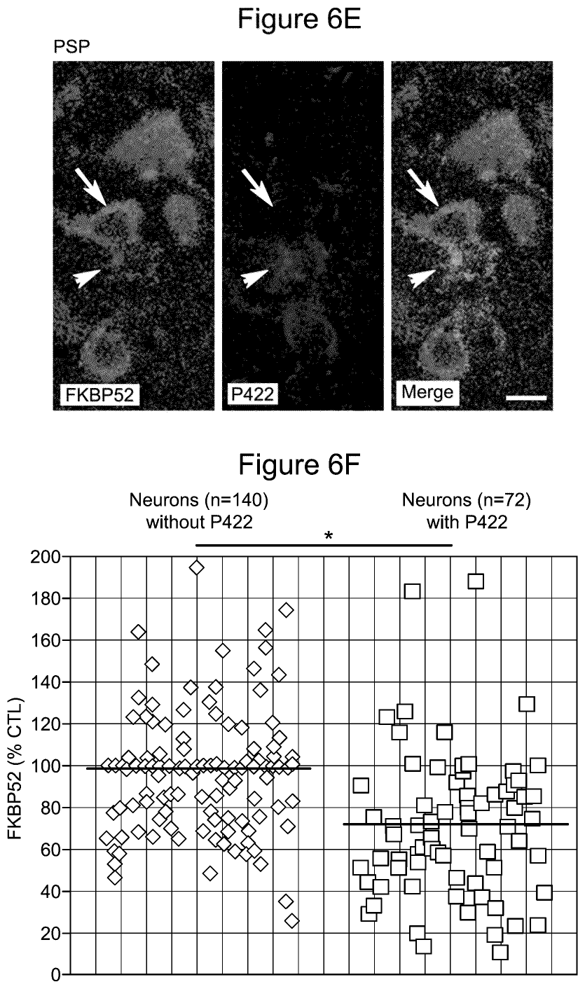

Tau-D421 and Tau-pS422 lysosomal localization in different tauopathies. The inventors report the association of FKBP52 with two early pathological Tau forms, the caspase-cleaved Tau-D421 and the Tau-pS422 phosphorylated at serine 422 in the ALP of AD, PSP and familial FTLD-Tau neurons. However, some studies propose a protective effect for both Tau-pS422 and Tau-D421. This discordance might be explained by the possibility that Tan forms, not deleterious in physiologic quantities, might abnormally accumulate in affected neurons through inefficient degradation, becoming pathogenic. It has been reported that Tau-D421 is preferentially degraded through autophagy supporting our observations of its lysosomal localization, while Tau phosphorylation at Serine 422 inhibits Tau-D421 formation preventing Tau autophagic degradation. Nevertheless, the inventors localize Tau-pS422 in the ALP which could suggest that the inhibition of caspase-induced Tau cleavage by S422 phosphorylation might be removed locally in the endo-lysosomal environment allowing cathepsin cleavage of Tau in order to facilitate the autophagic degradation of its fragments. Indeed, while Tau-D421 immuno-labeling often presents a vesicular/lysosomal aspect (FIGS. 3 and 4; Tau-pS422 immunoreactivity exhibits mingled NFTs, pre-NFTs and vesicular/lysosomal patterns in each tauopathy the inventors studied (FIGS. 5 and 6). This observation agrees with other studies showing the infrequent presence of Tau-D421 positive neurofilamentous Tau aggregates in AD, familial FTLD-Tau and in PSP brain neurons. A study even reports the absence of Tau-D421 signals in PSP brains, nevertheless the inventors detect in PSP brain neurons the same vesicular/lysosomal aspect of Tau-D421 immunoreactivity they observed in AD and familial FTLD-Tau brains suggesting that this kind of Tau caspase and/or lysosomal processing might occur in PSP neurons and contribute to Tau pathogenesis. They do not detect a significant expression of Tau-D421 in the hTau-P30IS mouse model as previously reported whereas they detect Tau-pS422 in rare mouse spinal cord neurons at 1 month of age. This difference in Tau-D421 deposition between the hTau-P30IS mouse model and the familial FTLD-Tau patients, both tauopathies exclusively caused by the human Tau gene mutation, supports the idea that caspase-cleavage of Tau at Asp421 is not an essential step towards neurodegeneration at least in pathologies linked to genetic dominant Tau mutations.

FKBP52 expression is decreased in neurons with different Tau deposits in different tauopathies. The presence of residual FKBP52 associated with pathological Tau in the lysosomal environment of AD, familial FTLD-Tau, PSP and in the hTau-P301S mice neurons suggests a common mechanism involved in FKBP52 decrease, potentially impacting on the pathologic process. The constant presence of FKBP52 in lysosomes and its constant association with early pathologic forms of Tau in lysosomes of affected neurons suggests that its decrease might be mainly linked to lysosomal processing. Also, the inventors have previously demonstrated the presence of extracellular release of FKBP52 in SH-SY5Y neurons under Tau-induced proteotoxic stress. It is therefore possible that different mechanisms might contribute to FKBP52 decrease in neurodegenerative diseases, albeit with a different impact. However, they also detect by IHC a little FKBP52 immunoreactivity embedded in big NFTs, thus a possible sequestration of this immunophilin in NFTs cannot be excluded, such a sequestration mechanism has been described for Pint, another peptidyl-prolyl cis/trans isomerase deregulated in AD neurons. The significant FKBP52 depletion in neurons bearing different pathologic Tau deposits (Tau-pS422 and Tau-AT180) in the frontal cortex of AD Braak VI, familial FTLD-Tau and PSP patients, without directly causing neurodegeneration, might nevertheless represent an aggravating consequence. Indeed, they have previously shown in dorsal root ganglion (DRG) neurons from hTau-P30IS mice that FKBP52 deficiency impacts on ALP functions triggering insoluble Tau accumulation under protracted Tau-induced proteotoxic stress. IHF analysis of brain neurons from patients with different tauopathies shows a range of FKBP52 decrease of approximately 27-34% in Tau-pS422-positive cells and 30-34% in Tau-AT180-positive cells. They also find a comparable FKBP52 decrease in the spinal cord neurons of Tau transgenic mice with pathological Tau deposits regardless of the absence of Tau-D421 deposition. Thus, the association of FKBP52 decrease and Tau deposition is not specifically related to Tau-D421 accumulation. All these observations support that FKBP52 decrease, as a consequence of Tau dysfunction and accumulation, might contribute to Tau pathogenesis in familial and possibly in sporadic tauopathies.

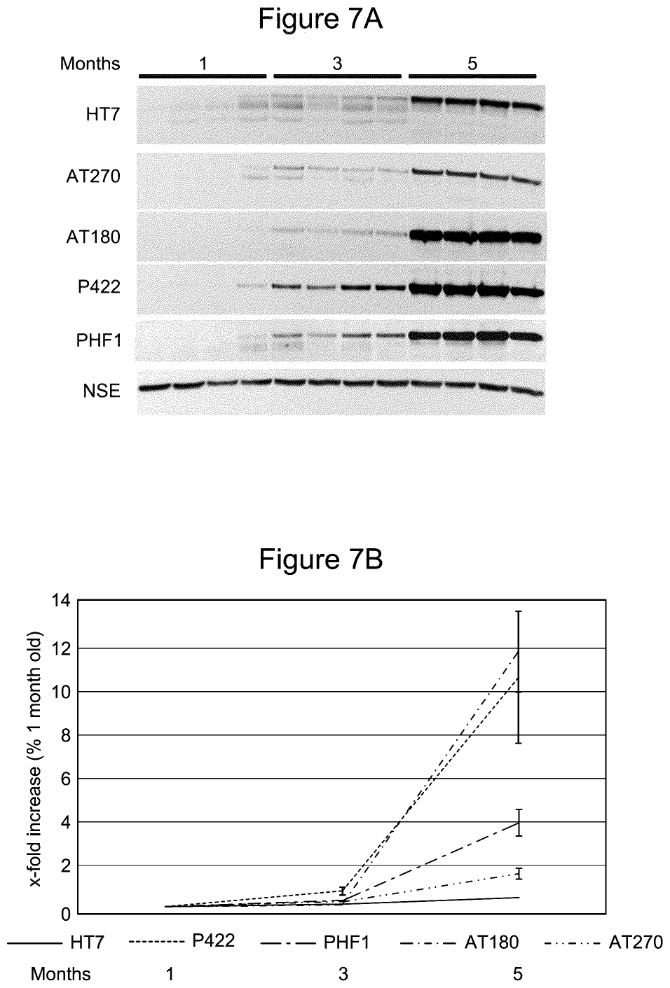

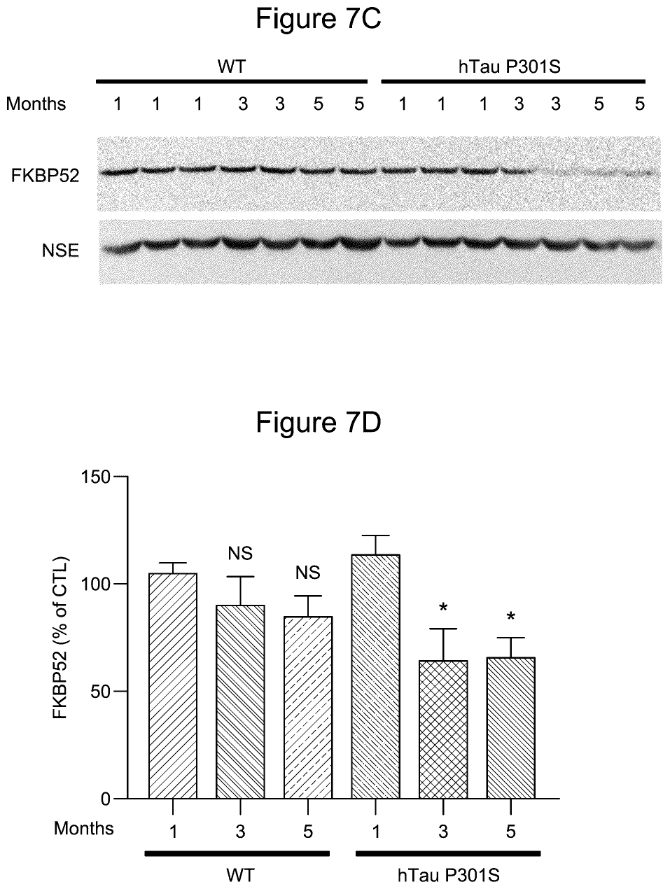

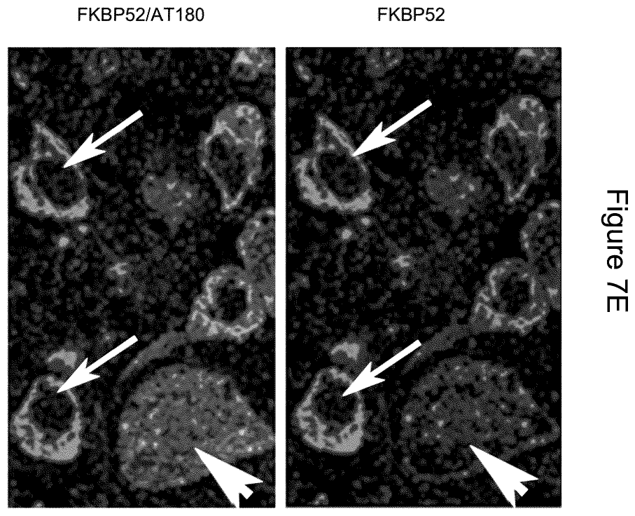

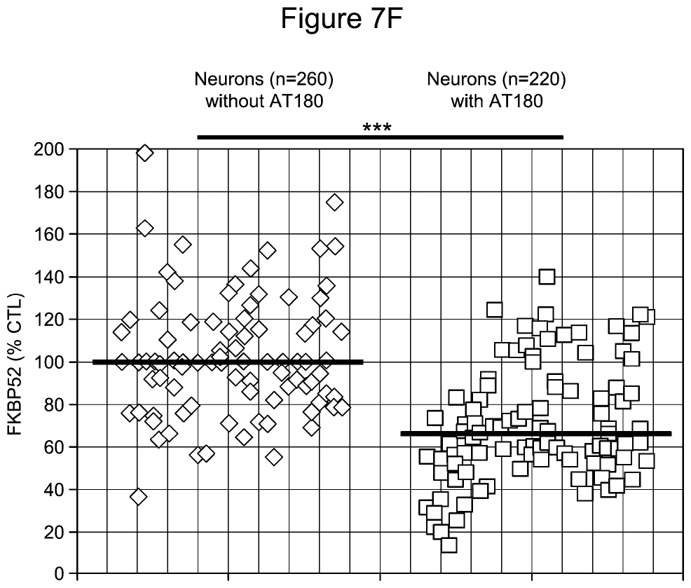

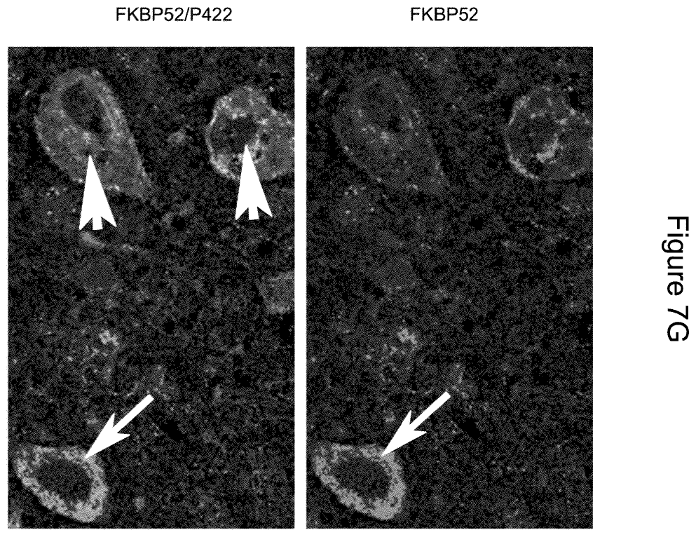

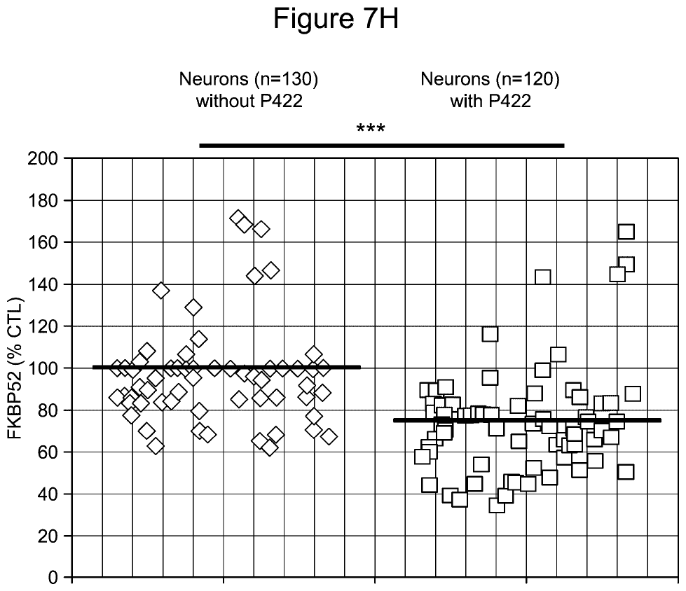

The inventors detected a FKBP52 decrease in Tau-pS422-positive neurons in scarcely affected brain regions from AD Braak IV patients and in the spinal cords neurons of 1 month old, symptomless hTau-P301S transgenic mice (FIG. 7). As Tau-pS422 is considered as an early tauopathy marker these findings seem to suggest that FKBP52 decrease also happens very early and in regions that are scarcely affected at such an initial stage of disease progression. The percentage values of FKBP52 depletion measured by IHF were lower than those observed by Western-blot analysis showing a decrease of ˜54% and ˜70% in the frontal cortex of familial FTLD-Tau (FIG. 3) and in AD Braak VI patients respectively. The different results between the techniques might be partly explained by the effects of formalin fixation on the preservation of epitopes revealed by IHF. This discordance might also be due to the fact that double labeling experiments for image analysis detect the deposits of one specific pathologic Tau form and fail to detect other type of Tau aggregates in adjacent neurons. This would obviously impact on the overall calculated FKBP52 decrease, without negating the significant reduction of FKBP52 in affected neurons. Despite these differences, the inventors constantly observe a decrease of FKBP52 levels in pathological neurons. A minor to moderate decrease of FKBP52 might be in itself sufficient to induce some detrimental effects on Tau function and accumulation over time as previously observed in DRG neurons from hTau-P301S mice. They also observe a strong heterogeneity of FKBP52 expression by Western-blot in frontal cortex samples from PSP patients in contrast to cell by cell IHF analysis, this difference might be explained by the pathological heterogeneity of PSP and the variations in the severity of frontal cortex neuronal involvement among patients.

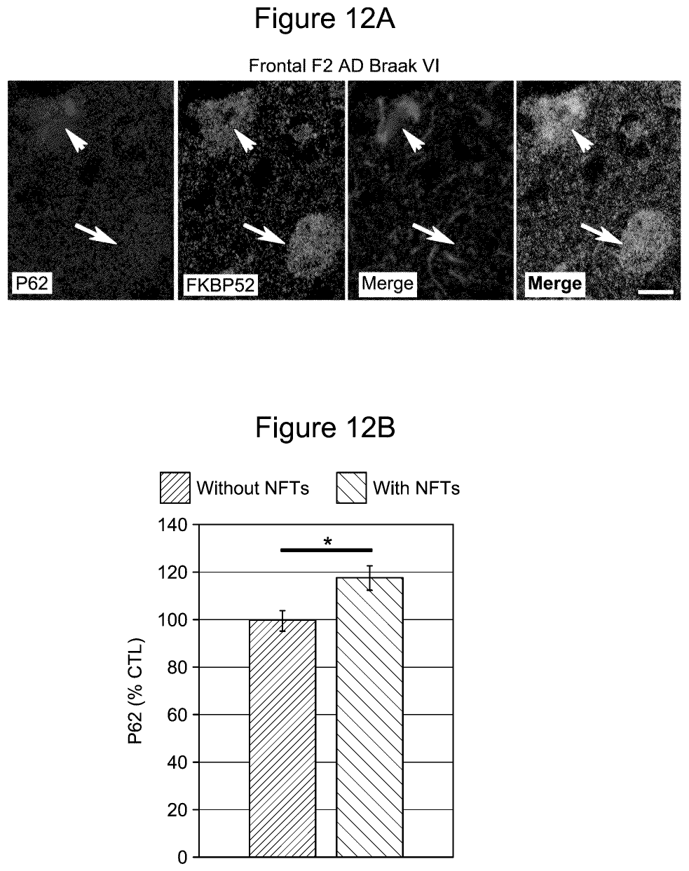

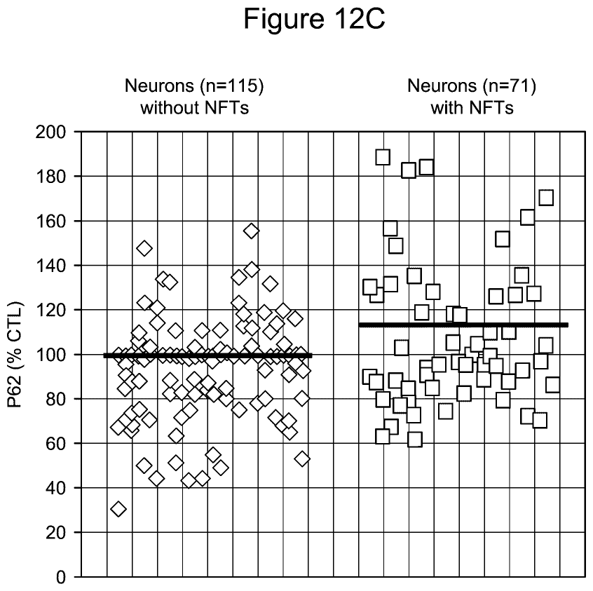

It has been recently shown that FKBP52 overexpression accelerates hippocampal neuronal loss and memory defects in a Tau transgenic mouse model and the study in question evokes the possibility that the decrease of FKBP52 expression in AD brains is due to the death of neurons expressing high FKBP52 levels during Tau pathogenesis. However, the inventors show by image analysis after multiple labeling of AD Braak VI frontal cortex samples that FKBP52 decrease in neurons with Tau deposits is not associated with the decrease of P62, another neuronal protein, whose levels are comparable to those found in non-affected adjacent neurons, excluding an effect of protein leakage from damaged or dying cells (FIG. 12). Nevertheless, an early rapid phase of neuronal FKBP52 increase preceding a successive slower decline cannot be totally excluded, especially as the distribution of FKBP52 expression values in the different scatter diagrams appears to be heterogeneous (See Figures). Notably, they do not detect an increase of FKBP52 expression levels in spinal cords of hTau P301S mice different ages by Western-blot analysis (FIGS. 7C and 7D).

To summarize, the inventors showed a consistent decrease of neuronal FKBP52 in the frontal cortex of different tauopathies such as PSP, familial FTLD-Tau and AD. Additionally, they show by image analysis that FKBP52 decrease happens concomitantly with the apparition of some pathological Tau isoforms including the early Tau-pS422 in spinal cord neurons from hTau-P301S mice. They also find that the FKBP52 decrease starts early during the pathologic process occurring in rare Tau-pS422 positive neurons in marginally affected frontal lobes of AD Braak IV brains and in the spinal cords of symptomless one month-old hTau-P301S mice. These observations lead us to think that the FKBP52 decrease in the brains of patients with some neurodegenerative diseases might be a new potential early marker of pathologic progression. Several imaging probes for the non-invasive detection of pathologic Tau aggregates are currently used in positron emission tomography (PET). However, actual Tau probes do not well discriminate between early and late disease stages and have difficulty in detecting Tau deposition in non-AD tauopathies possibly owing to post-translational events and different NFTs maturation that might remove ligand binding sites and influence PET imaging results. Besides, the new generation probe Flortaucipir shows off-target binding especially in hippocampus and basal ganglia, which might hinder its use in conditions such as early stage AD and Corticobasal degeneration. Thus, the detection of the prodromal decrease of a predominantly neuronal protein such as FKBP52 in the brain might helpfully contribute to the early diagnosis of some neuropathological diseases. Also, as FKBP52 plays an important role in cellular signaling and possibly in Tau clearance, these data suggest that preventing FKBP52 decrease or restoring its normal expression in tanopathies including PSP, familial FTLD-Tau and AD provides a foundation for new therapeutic approaches.

### The Following Materials and Methods were Used for the Work Reported in Example 4

Antibodies and chemicals. The concentrations and the provenance of the antibodies used in this study are listed in Table 1. The inventors used antibodies against Tau and different phosphorylation sites of Tau such as AT180 (Thr231 and Ser235), AT270 (Thr 181) and HT7 (anti-human and bovine Tau) (ThermoFisher). The caspase-cleaved Tau-D421 antibody (Tau-C3) was from SantaCruz Biotechnology. The phosphorylated Tau-pS422 antibody (2H9) was from 4BioDx. The anti-Tau PHF1 antibody was a king gift from Pr. P. Davies (Albert Einstein, College of Medicine, NY, USA). The FKBP52 antibodies used were from Abcam (EPR6618) or Enzo (EC1). For colocalization experiments, they used antibodies directed against the endo-lysosomal system: Cathepsin D (lysosomal marker, Santa Cruz Biotechnologies), LC3 (autophagosome marker, Novus) and Rab7 (late endosomes marker, Abcam). Donkey anti-mouse, rabbit and goat IgG (H+L) secondary antibodies labeled with Alexa Fluor 488, 555, 546 and 647 by Life Technologies were purchased from Molecular Probes.

Human brain samples. Brain samples from individuals without neurological disorders and from AD, PSP and familial FTLD-Tau patients of comparable age and post mortem delay were obtained from the Brain Bank GIE NeuroCEB (Table 2). The samples were collected on behalf of a Brain Donation Program funded by a consortium of Patients Associations: ARSEP (association for research on multiple sclerosis), CSC (cerebellar ataxias), France Alzheimer and France Parkinson. The consents were signed by the patients themselves or their next of kin in their name, in accordance with French Bioethical Laws. The Brain Bank GIE NeuroCEB (Bioresource Research Impact Factor number=BRIF BB-0033-00011) has been authorized to provide samples for research by the Ministry of Higher Education and Research (agreement AC-2013-1887). Prior to histological analysis, a brain hemisphere, randomly left or right, was fixed in buffered 4% formaldehyde. Samples from multiple regions were embedded in paraffin and cut at Sum thickness. For diagnosis and staging, sections of affected brain regions were stained with hematoxylin-eosin, and immunostained with anti-Aβ (Dako, 6F/3D clone, 1:200), anti-AT8 (ThermoFisher, AT8, 1:500), anti-α-synuclein (Zymed, LB 509 clone, 1:250) monoclonal antibodies, and with an anti-ubiquitin antibody (polyclonal Dako, 1:500). AD cases were diagnosed according to the National Institute of Aging and Reagan Institute Criteria (1997). Control cases without known neurological disorders were systematically analyzed and used as negative control for pathological markers. For biochemical analysis, post-mortem tissues were snap-frozen in liquid nitrogen and stored at −80° C.

Animal models. Brains and spinal cords were collected from transgenic mice homozygous for the human Tau-P301S mutation at different ages. This mouse line, expressing the 383 amino acid isoform of human MAPT, displays a tauopathy phenotype characterized by severe paraparesis and a significant reduction in the number of motor neurons in the spinal cord at 5 months of age. Approval of the Inserm regional committee for animal experimentation, as established by the Scientific Research Ministry on January 1992, was previously obtained. Obligations required by the no 86/609 European directive for use of laboratory animals were also observed. Mouse brains and spinal cords from animals aged from 1 to 5 months were fixed in buffered formalin for 24 hours maximum, paraffin-embedded and sectioned prior to immunolabeling experiments.

Immunohistofluorescence (IHF). Formalin fixed paraffin-embedded brain samples were sectioned at 8 μm thickness. Sections were mounted on Superfrost+slides. After paraffin removal with three successive baths in xylene substitute for 5 minutes and three baths in absolute alcohol, the sections were rehydrated in 70% alcohol, rinsed in demineralized water and subjected to standard microwave antigen retrieval in pH6 Citrate buffer. Sections were then incubated with SEA BLOCK Blocking Buffer (ThermoFisher, Rockford, II, USA) for 30 minutes prior to primary antibody incubation overnight at 4° C. in a humid chamber. After PBS washing, the sections were then incubated with appropriate donkey anti-mouse, goat, or rabbit secondary antibodies labeled with AlexaFluor 488, AlexaFluor 555 or 546, or AlexaFluor 647 at a concentration of 1.6 μg/ml for 90 minutes at room temperature. Pre-immune mouse, rabbit and goat immunoglobulins were used in place of the primary antibodies as negative controls. For double and triple labeling experiments, overnight primary antibody incubations were repeated serially with successive appropriate secondary antibody incubations as they found that specific signal intensity was greater than using simultaneous application of primary and then secondary antibodies mixtures.

After antibody incubation and washing, lipofuscin autofluorescence on human brain sections was quenched with Millipore Autofluorescence Eliminator Reagent (Millipore, Cal USA) according to the manufacturer's instructions. A DAPI (4′,6-diamidino-2-phenylindole) enriched mounting medium (Abcam) was used and provided a nuclear counterstain. Slides were examined with a Leica TCS SP8 confocal microscope with Z-stack imaging.

Western-blotting. For each case studied (Table 2), a portion of medial frontal cortex (F2) was homogenized at 4+C using a Dounce homogenizer (glass-Teflon) in 5 volume (w/v) of buffer consisting of: Tris 10 mM, saccharose 0.32 M, and DTT 1 mM at pH 7.4 with “Complete” protease inhibitor cocktails (Roche). Homogenates were centrifuged 5 min at 1000 g and the protein estimation of the upper fraction was performed using a BCA protein assay kit (Pierce). Each samples were analyzed on a 10% SDS Page gel and transferred onto iBlot™ Gel Transfer membranes (Invitrogen). Following transfer, membranes were incubated overnight with appropriate antibodies [FKBP52, mouse monoclonal EC1 (1:1000) (Enzo Life Sciences) and a Rabbit polyclonal HRP conjugated GAPDH (1:1000) (Abcam) rather than Actin for the loading control as recommended. Images were recorded with the GeneGnome5 (Syngene). Quantification was performed using Genetools analysis software (Syngene).

Image analysis. The fluorescence profile was evaluated on a single plane of each confocal image using FIJI freeware image analysis program. The intensity of fluorescence in the green and red channels was measured along a line segment. To assess FKBP52 immunoreactivity in neurons with or without AT180, Tau-D421, Tau-pS422 positive NFTs, or P62 labeling, confocal image were analyzed with the FIJI image analysis program.

Statistical analysis. Statistical analysis was performed using one-way variance analysis (Student's t test) comparing the mean of percentages (#S.E.M.) of at least four independent experiments. Level of significance: N. S. not significant; P values of <0.05 were considered statistically significant. The software GraphPad Prism 8 was used to determine the statistical analysis.

### Results of Work Described in Example 4

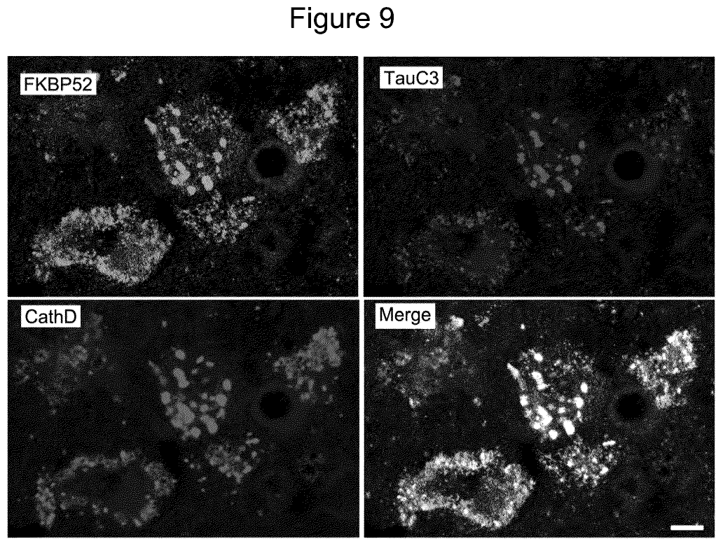

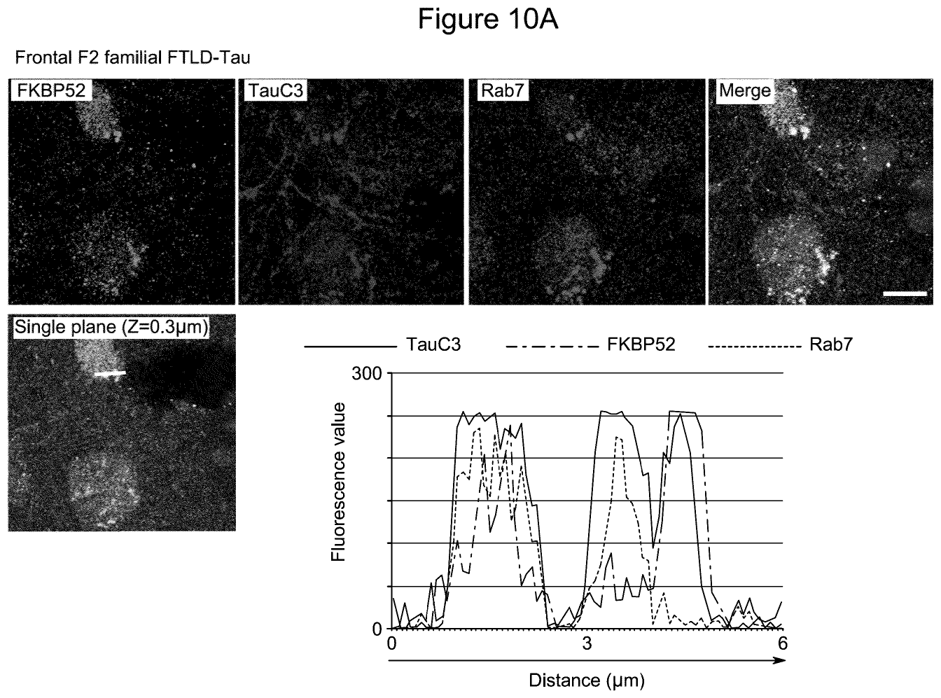

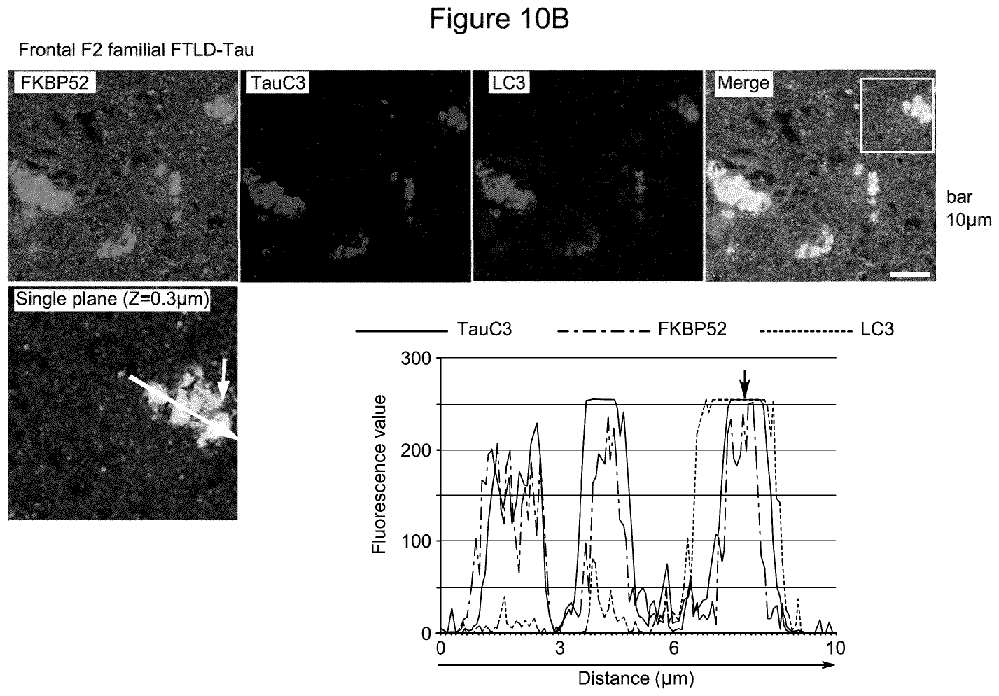

FKBP52 partially colocalizes with truncated Tau-D421 in the autophagy endo-lysosomal pathway (ALP) of familial FTLD-Tau brain neurons and its decrease is related to NETs deposition. IHF analysis performed in familial FTLD-Tau brains showed the colocalization of the FKBP52 signal with cathepsin D, an endosomal/lysosomal marker, as previously observed in AD and normal brain neurons. It also showed a decrease of FKBP52 expression in isocortical neurons with pathological AT180 Tau deposits (FIG. 3A) when compared to adjacent neurons without detectable deposits, analogously to what previously described in AD Braak VI. Residual FKBP52 in familial FTLD-Tau brain neurons was frequently localized into abnormally large endosomal/lysosomal vesicles (FIG. 3A, see magnifications) and FIG. 9). The fluorescent levels of FKBP52 immunoreactivity were examined in 108 neurons per sample in 4 familial FTLD-Tau cases (total: 432 neurons), and in 114 neurons per sample in 4 normal controls (total: 456 neurons). The mean value of the FKBP52 signal per cell was 100%+/−10.5% [SEM] for the normal controls, 57.8%+/−9.1% for the FTLD-Tau cases; the difference was highly significant (**p<0.01; FIG. 3B). The signal per cell was, as a mean, inferior by 42% in familial FTLD-Tau neurons confirming the decrease of FKBP52 expression levels previously observed by Western-blotting on total frontal cortex homogenates (46.1%+/−7.8% [SEM]; FIGS. 3C and 3D). While FKBP52 was decreased in AT180 immuno-positive neurons in familial FTLD-Tau samples (FIG. 3E, arrowhead), neurons without apparent Tau deposition exhibited FKBP52-positive endosomes/lysosomes similar to those seen in healthy control brains (arrow). In familial FTLD-Tau brains, the fluorescent level of FKBP52 immunoreactivity in neurons with AT180-positive NFTs was 69.5% (+/−4.1% [SEM]) of the value found in neurons without NFTs (96.4%+/−1.6%). A mean of 35 neurons without NFTs and 25 NFTs-bearing neurons per case was analyzed from 4 familial FTLD-Tau cases, and the difference was highly significant (t=6.13, df=n1+n2−2=6; ***p<0.001). The levels of FKBP52 labelling from each of the different neurons studied are presented in a scatter diagram (FIG. 3F) Triple labeling experiments demonstrated that FKBP52, cathepsin D, and caspase-cleaved Tau-D421 (TauC3 antibody) immunoreactivities colocalized in FTLD-Tau neurons as previously observed in AD neurons (FIGS. 3G and 3H, see arrow in fluorescence profile, Z=0.3 mm). This colocalization was also observed into large endo-lysosomal vesicles (FIG. 9). FKBP52 and Tau-D421 also colocalized in Rab7-positive late endosomes and LC3-positive autophagic vesicles (FIG. 10). Our findings demonstrate that residual FKBP52 is detected along with an early pathological and caspase-cleaved Tau form in the ALP of FTLD-Tau neurons and suggest that the pathologic decrease of FKBP52 is associated with NFT apparition in affected neurons.

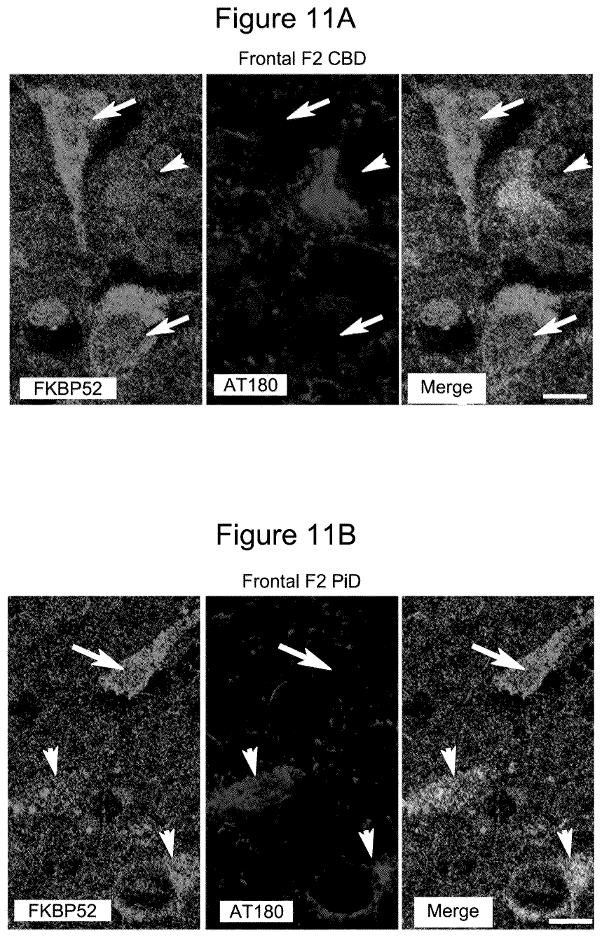

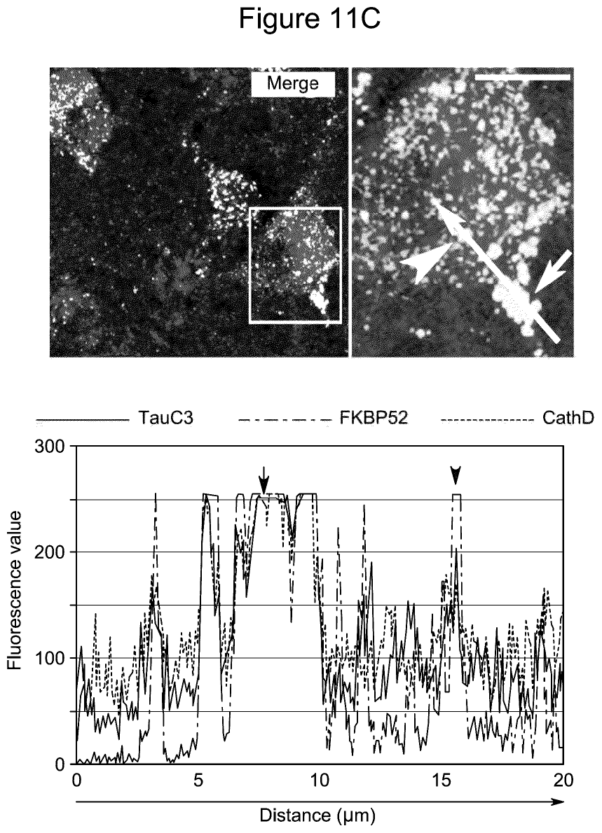

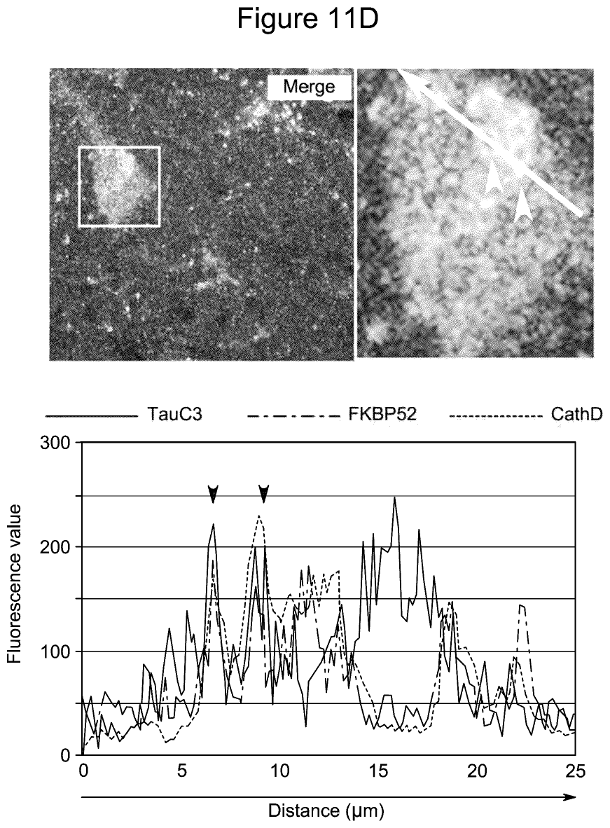

Neuronal FKBP52 decrease and its colocalization with truncated Tau-D421 in the ALP are also detected in another human tauopathy such as PSP. In order to check if FKBP52 expression was also modified in the brains of patients with other tauopathies, the inventors studied frontal cortex tissue samples from S PSP patients. Western-blot analysis of FKBP52 in PSP brain homogenates showed a non-statistically significant decrease (48.3%+/−17.5%) (1=2.07, df=6; p=0.0837, FIG. 4A) very likely resulting from a strong heterogeneity of FKBP52 expression between PSP samples (n=5; 7.6 to 82%). This difference might be explained by the pathological heterogeneity of PSP and the variable severity of Tau pathology in the frontal cortex between patients. In contrast, cell by cell IHF analysis of AT180 NFTs-bearing neurons in the frontal cortex of PSP patients showed a significant decrease of FKBP52 (FIG. 4B, arrowhead) compared to neurons without apparent Tau deposition (FIG. 4B, arrow). The fluorescent levels of FKBP52 immunoreactivity in PSP frontal cortex neurons with AT180 deposits were 66.6% (+/−4.43% [SEM]) of the value found in neurons devoid of deposits (104.4%+/−1.96% [SEM]). A mean of 47 neurons without NFTs and 20 NFTs-bearing neurons per case was analyzed from five PSP cases resulting in a very significant difference (t=7.8, df=8; **, p=0.0015). The levels of FKBP52 labelling from each of the different neurons studied are presented in a scatter diagram (FIG. 4C). They observed by IHF analysis a comparable interrelation between the decrease of FKBP52 and the presence of AT180-Tau deposits in frontal cortex neurons from CBD (n=2) and PID (n=2) brains but the number of samples for both tauopathies was too low to determinate the significance of these observations (FIG. 11A). As previously observed in AD and familial FTLD-Tau neurons, Tau-D421 immunoreactivity also colocalizes with FKBP52 and CathD in PSP (FIGS. 4D and 4E, see arrow in the fluorescence profile, Z=0.3 mm), CBD and PiD neurons (FIG. 12B). Altogether, these findings suggest that neuronal FKBP52 decrease and its colocalization with Tau-D421 in the ALP is not specific to a single tauopathy as it is significantly detected in the brains of AD, familial FTLD-Tau and PSP patients.

The abnormal decrease of FKBP53 and its colocalization with pathologic Tau forms in the ALP of affected neurons in AD, FTLD-Tau and PSP frontal cortex is not restricted to cells with caspase-cleaved Tau-D421 deposits. Caspase activation is often associated with apoptotic signals. The inventors detected the caspase cleaved Tau-D421 in association with FKBP52 in the ALP of affected neurons. This might suggest that the colocalization they observed is restricted to a population of apoptotic neurons. They thus decided to extend our study and focus our attention on another pathologic Tau isoform. Tau cleavage by Caspases at serine 421 is inhibited by phosphorylation at serine 422 (Tau-pS422), which prevents Tau autophagic clearance, triggering Tau-pS422 accumulation and possibly contributing to the escape of neurons from acute apoptotic death. It has been reported that Tau-pS422 is not preferentially degraded by autophagy unlike Tau-D421. They detected by IHF analysis a focal Tau-pS422 localization in the ALP of AD, familial FTLD-Tau and PSP affected neurons (FIG. 5). Also, they observed a colocalization of FKBP52 with both Tau-pS422 and Cathepsin D suggesting that the association of FKBP52 with Tau in the ALP in these tauopathies is not restricted to potentially apoptotic neurons bearing caspase-cleaved Tau-D421 (FIG. 5, see arrowheads in fluorescence profiles, Z=0.3 mm). Tau-pS422 accumulation is considered an earlier Tau pathologic event than formation of Tau-D421 aggregates. They thus measured by IHF analysis the fluorescent levels of FKBP52 immunoreactivity in neurons with Tau-pS422 deposits in AD Braak VI, familial FTLD-Tau and PSP cases (FIG. 6). Tau-pS422 positive neurons in AD Braak VI, familial FTLD-Tau and PSP cases exhibited a significant decrease of FKBP52 signal (FIGS. 6A, 6C and 6E; arrowheads) compared with neurons without apparent Tau-pS422 deposition (arrows). The fluorescent levels of FKBP52 immunoreactivity in neurons with Tau-pS422 deposits were 72% (+/−5.2%), 66% (+/−4.3%) and 73% (+/−7%) of the value found in neurons without aggregates (90%+/−2.6%, 98%+/−1.5% and 98%+/−2.9%) respectively in AD Braak VI, familial FTLD-Tau and PSP brains (FIGS. 6B, 6D and 6F). A mean of 48 neurons without Tau-pS422 aggregates and 43 neurons bearing aggregates were analyzed from frontal cortex samples of 13 AD Braak VI patients showing a statistically significant decrease of FKBP52 signals in affected neurons (t=2.54, df=24; *, p=0.0177; FIG. 6B). Also, the analysis of a mean of 46 neurons without Tau-pS422 aggregates and of 40 neurons with Tau-pS422 deposits per patient from 4 familial FTLD-Tau frontal cortex samples evidenced a highly significant difference in FKBP52 signals between neurons with and without pathological deposits (t=6.97, df=6; ***, p=0.0004; FIG. 6D). Analogously, the analysis of a mean of 28 neurons without Tau-pS422 aggregates and 15 aggregate-bearing neurons per sample from 5 PSP patients, showed a statistically significant decrease of FKBP52 in neurons with Tau-pS422 deposits (t=2.9, df=8: *, p=0.0191; FIG. 6F). Overall, these results show that FKBP52 decrease is detected both in Tau-D421 and Tau-pS422 positive neurons from the frontal cortex of AD, familial FTLD-Tau and PSP patients and suggest that the pathological decrease of FKBP52 they describe is not restricted to Tau-D421 formation and accumulation in pathologic neurons from these different tauopathies.

FKBP52 expression decreases in affected spinal neurons of hTau-P301S mice in parallel with the deposition of pathological Tau and independently of Tau-D431 formation and accumulation. Next, the inventors checked whether the abnormal expression and subcellular localization of FKBP52 observed in familial FTLD-Tau brain neurons could be also detected in a transgenic mouse model of FTLD-Tan (hTau-P301S mice;). At about 5 months of age, the homozygous hTau-P301S mice display a tauopathy phenotype characterized by severe paraparesis and the presence of abundant intraneuronal Tau deposits in the brain and in the spinal cord. Western-blot analysis of spinal cords from 1 to 5 months-old hTau-P30IS mice showed a progressive accumulation of human Tau proteins, specifically recognized by the HT7 antibody, along with the detection of different pathological phosphorylated Tau isoforms, recognized by several antibodies such as AT180 and Tau-pS422, already significantly present at 3 months of age (FIGS. 7A and 7B). Others had previously reported a very low or absent caspase activity and a very low expression of Tau-D421 in hTau-P30IS mice, which made it particularly suitable to exclude the possible influence of neuronal apoptosis on our results. Although several caspase-cleaved Tau-D421 positive neurons were detected in familial FTLD-Tau brain samples, only rare Tau-D421 positive neurons were present in the spinal cords and brains of hTau-P301S mice as previously described. Despite the absence of this pathological Tau form, Western-blot analysis of total spinal cord homogenates from mice of different ages showed a relevant decrease of FKBP52 already at 3 months (64.5%+/−13.7% [SEM]) compared with 1 month old mice (112%+/−9.8% [SEM]; FIG. 7D) with little variation of its expression until S-months (65%+/−9.7% [SEM]). These results confirm our previous observations showing that FKBP52 decrease is independent of Tau-D421 accumulation but nevertheless happens in concomitance with the apparition of pathological Tau deposits (FIGS. 7A and 7C). IHF analysis of the spinal cord of 5 months old mice supports our observations about the FKBP52 decrease both in AT180 and Tau-pS422-positive spinal neurons (FIGS. 7E-7H). The fluorescent levels of FKBP52 immunoreactivity in neurons with Tau aggregates are 70.9% (+/−1.1%) and 70.3% (+/−3.5%) of the value found in neurons without aggregates (102.7%+/−1.7%) and (100.1%+/−1.8%) respectively in AT180 and Tau-pS422 positive neurons (FIGS. 7F and 7H; [SEM]). A mean of 52 neurons without Tau AT180 aggregates and of 44 aggregate-bearing neurons per sample were analyzed from 5 spinal cords of 5 months-old mice, and the difference was statistically significant (t=12.47, df=8; ***, p=0.0001; FIG. 7F). Analogously, a mean of 26 neurons devoid of Tau-pS422 aggregates and of 24 neurons with aggregates per sample were analyzed from 5 spinal cords of 5 months-old mice, and the difference was also statistically significant (t=6.73, df=8; ***, p=0.0001; FIG. 7H). Altogether, in the hTau-P30IS mouse model of tauopathy, they observed an early pathological decrease of FKBP52 in the spinal cord neurons that occurred in parallel to Tau accumulation. These results are analogous to the FKBP52 decrease in neurons with pathologic Tau deposits they detected in the brains of patients with a pure tauopathy, such as familial FTLD-Tau, thus corresponding to an early decrease of FKBP52 at the preclinical stage in familial FTLD-Tau patients as well as in other human tauopathies.

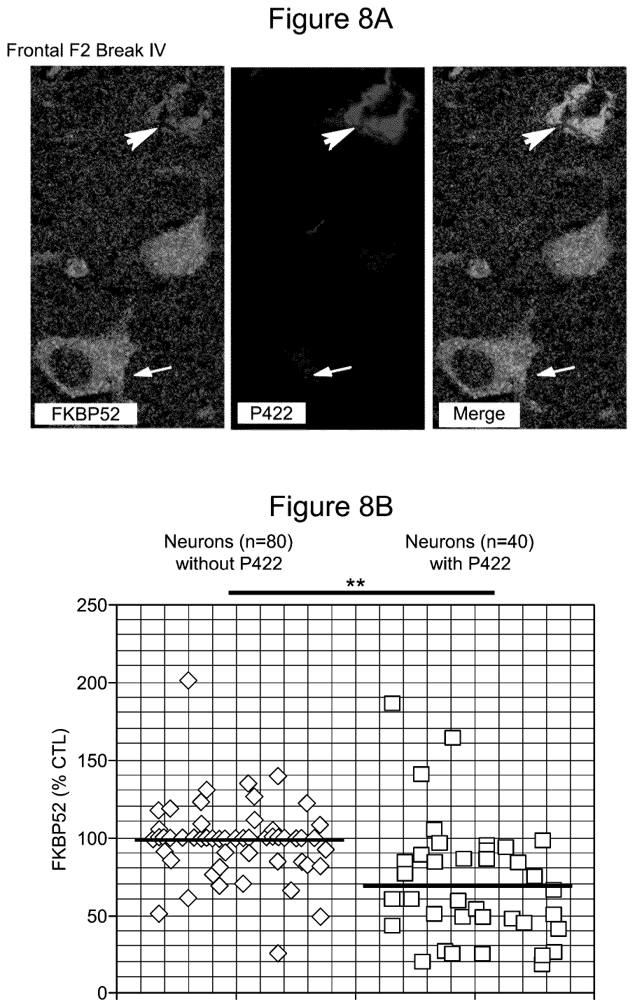

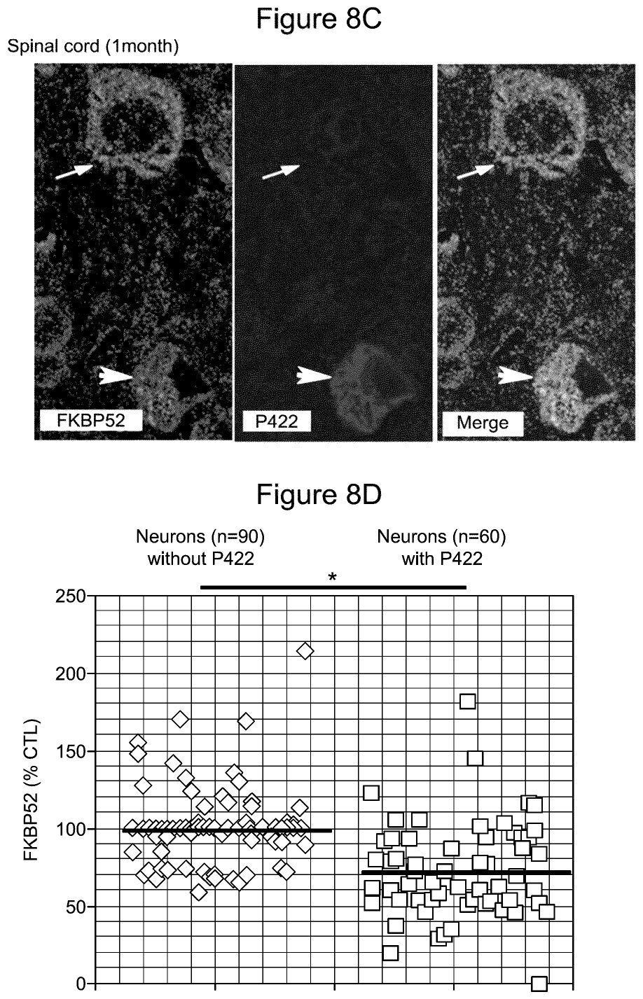

FKBP52 is decreased in pathologic neurons both from marginally affected AD Braak IV brain regions and from the rachis of symptomless one month-old hTau-P301S mice. The previously described results prompted us to investigate the levels of intraneuronal FKBP52 at a very early stage of disease progression. The inventors thus examined the scarcely affected frontal cortex of AD patients with Braak IV stage (n=5) and the spinal cords of symptomless one month-old hTau-P301S mice. Even if these samples comported few Tau-pS422 positive neurons, they were nevertheless able to analyze by IHF the variations of FKBP52 fluorescent signals in these rare cells (FIG. 8). Both Tau-pS422 positive neurons in AD Braak IV frontal cortex samples and in the spinal cord of symptomless one month-old hTau-P301S mice exhibited a significant decrease of FKBP52 (FIGS. 8A and 8C) compared to neurons without Tau-pS422 deposition. The fluorescent level of FKBP52 immunoreactivity in neurons with Tau-pS422 aggregates was 72.6% (+/−6.8%) and 72.5% (+/−5.9%) of the value found in neurons without Tau-pS422 aggregates (100.1%+/−3.1% and 99.9%+/−5.5%) respectively in AD Braak IV and in the spinal cords of one month-old hTau-P301S mice (FIGS. 8B and 8D). A mean of 16 neurons without apparent Tau aggregates and of 8 Tau-pS422 aggregate-bearing neurons per patient was analyzed from 5 AD Braak IV frontal cortex samples, and the difference was highly significant (t=3.65, df=8; **, p=0.0065; FIG. 8B). The difference in FKBP52 expression was also significant when a mean of 18 neurons without apparent Tau aggregates and of 12 neurons with Tau-pS422 aggregates per sample was analyzed from the spinal cords of three 1 month-old hTau-P301S mice (t=3.37, df=4; *p=0.0279; FIG. 8D). The expression of FKBP52 measured by Western-blot analysis in the spinal cord of one month-old hTau-P301S mice is similar from that observed in wild-type mice of the same age (FIGS. 7C and 7D), in contrast with our findings obtained by IHF and cell by cell image analysis showing a significant decrease of FKBP52 in the rare Tau-pS422 bearing neurons (FIGS. 8C and 8D). This difference is very likely due to the rarity of affected neurons at such an early stage of disease: indeed our results suggest that FKBP52 decrease has already started in these tissues but is limited to a low number of affected neurons while its expression is unchanged in the unaffected cells. Altogether, these experiments show that the neuronal decrease of FKBP52 happens as early as Tau-pS422 deposition is detectable in marginally affected regions of AD brains and in the spinal cord of symptomless one month-old hTau-P301S mice.

Terminology. The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

The terms “we” and “us” refer to the inventors or to the contributions of one or more inventors.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

### DETAILED DESCRIPTION OF THE INVENTION

Tauopathies. This term refers to a group of neurodegenerative diseases characterized by the abnormal accumulation add aggregation of tau proteins in the brain or nervous system leading to neuronal dysfunction and/or degeneration. A large number of neurodegenerative diseases, including Alzheimer Disease (AD), Pick's disease (PiD), progressive supranuclear palsy (PSP), corticobasal degeneration (CBD), and frontotemporal lobar degeneration due to the mutation of Tau gene (FTLD-Tau) are characterized by intra-neuronal aggregates of Tau, which are hallmarks of those disorders of the human brain now called tauopathies. Features of tauopathies include misfolding and aggregation of tau proteins which normally stabilize microtubules, formation of neurofibrillary tangles or NFTs composed of abnormal tau proteins and neuronal toxicity and degeneration due to disruption of normal tau function. The methods disclosed herein may treat one or more of the above-mentioned features. Other tauopathies are argyrophilic grain disease (AGD), globular glial tauopathy (GGT), and Primary age-related tauopathy (PART). Besides those tauopathies described above, the invention contemplates treatment of other diseases, disorders, or conditions associated with dysregulation or aggregation of tau protein function or tauopathies associated with various isoforms of tau protein including 3R, 4R or mixed 3R/4R tau isoforms. Treatment of conditions such as normal brain aging or tauopathy resulting from exposure to biological, chemical or pharmaceutical agents, including alcohol, cannabis, tobacco, cocaine, or other illegal or recreational drugs, which are characterized by at least a degree of tauopathy are also contemplated. In some embodiments, the method disclosed herein will reduce the severity of a tauopathy or its symptoms or decrease the incidence or degree of aggregation of Tau protein or of NFTs, for example, by 1, 2, 5, 10, 15, 20% or more.

Symptoms of tauopathies. The methods as disclosed herein may prevent, treat, ameliorate or reduce the severity of one or more symptoms of a tauopathy. Such symptoms include cognitive/behavioral disorders, such as problems with cognition, vision, language, executive function, and social cognition; changes in personality, social behavior and decision-making abilities; or dementia and memory impairment. Other symptoms include movement disorders such as Parkinsonism, apraxia, alien limb phenomena, and gait disturbances and freezing. Language disorders are also symptoms of some tauopathies and include non-fluent agrammatic primary or progressive aphasia, apraxia of speech. Other symptoms include psychiatric or amnesic symptoms, late-onset cerebellar ataxia and primary lateral sclerosis. Methods for objectively characterizing and quantifying these symptoms are known. For example, in tauopathies, regional topography of Tau pathology correlates well with clinical symptoms. In vivo molecular imaging using radiotracers provide useful techniques to diagnose the intensity of Tau accumulation and the progression of the tauopathy in the brain of suffering patients. The invention may provide significant relief for one or more symptoms, for example, a reduction in intensity, frequency or progression of such symptoms by at least 1, 2, 5, 10, 15, 20% or more as determined by objective or subjective medical diagnostic procedures known in the art.

FKBP52 (FK506 Binding Proteins of ˜52 kDa is a peptidyl-prolyl cis/trans isomerase implicated in the folding and function of its target protein. Besides its enzymatic activity, FKBP52 also displays a chaperone activity. While FKBP52 has mainly been described as a chaperone of steroid receptors, it is also involved in many other biological processes. FKBP52 is largely distributed and particularly abundant in the nervous system. The inventors have previously shown that FKBP52 prevents in vitro microtubule formation through its interaction with Tubulin and Tau and that FKBP52 is able to interact with the “PHF6” sequences of Tau by NMR experiments. Moreover, they have shown that FKBP52 levels are strongly decreased in the frontal cortex of AD and familial FTLD-Tau brains and that this decrease is highly correlated with the accumulation and aggregation of pathological Tau. Both mechanisms and timeline of this FKBP52 decrease in AD and other tauopathic brains are still unknown.

Peptides and Peptide-related products. These terms refer to peptides, peptide fragments such as subsections of a longer, full-length peptide, modified peptides such as peptides substituted with one or more D-amino acid residues or D-amino acid peptides, peptides incorporating non-natural residues such as beta-amino acids, methylation of backbone residues of a peptide, modification of the N-terminus or C-terminus of a peptide, including acetylation of the N-terminus or amidation of the C-terminus, peptide stapling to form or retain secondary, tertiary or quaternary structures, conjugation to targeting moieties such as transferin or insulin receptors to enhance Blood Brain Barrier (BBB) penetration, conjugation of C or N terminus to a fatty acid, or conjugation or linkage of a fluorescent dye to a peptide, or addition of an affinity tag to the N or C terminus. The peptides disclosed herein such as P35, and P50 may be administered as peptides or as any of the peptide products disclosed above, for example, P35 or P50 may be conjugated to a CPP or have their N or C terminals modified. The term “modified form” describes a peptide that has been chemically modified or that is structurally distinct from its natural form, for example, a modified form of the P35 or P50 peptide excludes the natural unmodified amino acid sequence of these peptide fragments but would include P35 or P50 peptides that have modified N or C termini, have modified peptide backbones (e.g., via methylation), or that are conjugated to other peptides or proteins such as P35-CPP or P50-CPP. The methods and compositions herein may be practiced or formulated with the unmodified peptides described herein, such as P35 or P50 or their active fragments, or, alternatively, with modified peptides or peptides such as P35 or P50 conjugated to other molecules, such as CPPs. In other embodiments, a modified peptide, such as a modification to P3S or P50 may have an N-terminal cysteine residue or a C-terminal cysteine residue, or both.

Peptide fragments or other constructs based on P35 or P50. In some embodiments the peptide or peptide product of the invention will consist or comprise a fragment or modified fragment of P35 or P50 peptides of sufficient length to protect or reduce the severity of a tauopathy, for example, a peptide fragment of P50 between 5, 10, 15, 20, 25, 30, 35, 40, 45 and 49 residues, or a fragment of P35 between 5, 10, 15, 20, 25, 30, and 34 residues in length.

In another embodiment, P35 or P50 may consist of 1, 2, 3, 4, 5, 6, 7, 8, 9, 10, 15, 20, 25 or 30 additional residues of the FKBP52 upstream from the N-terminal or downstream from the C terminal of P35 or P50. Peptide products with such additional residues typically will be adequate to protect or reduce the severity of a tauopathy.

In another embodiment, 1, 2, 3, 4, 5, 6, 7, 8, 9, 10, 15, 20, 25 or 30 additional peptide residues, which may or may not correspond to the adjacent residues in FKBP52, may be linked to either the N or C terminal of P35 or P50, for example, protein tags, CPP sequences, etc. Peptide products with such additional residues typically will be adequate to protect or reduce the severity of a tauopathy.

In another embodiment, 1, 2, 3, 4, 5 up to 20 residues P35 or P50 may be deleted or substituted with conservative or non-conservative amino acid residues. In another embodiment, I, 2, 3, 4, 5 up to 20 residues may be inserted into the internal sequences of P35 or P50. Peptide products with such additional modification typically will be adequate to protect or reduce the severity of a tauopathy.

Some non-limiting examples of the peptides or peptide products described herein are shown below.

P50
(SEQ ID NO: 2)
(AKPNEGAIVEVALEGYYKDKLFDQRELRFEIGEGENLDLPYGLERAIQ
RM)
P35
(SEQ ID NO: 1)
(AKPNEGAIVEVALEGYYKDKLFDQRELRFEIGEGE)
YARA-P50
(SEQ ID NO: 8)
(YARAAARQARA-P50)
YARA-P35
(SEQ ID NO: 9)
(YARAAARQARA-P35)

Cell penetrating peptides or CPPs are short peptides that assist intake or uptake of a peptide product (or nucleic acid) to which they are associated or covalently bound. CPPs may be cationic (e.g. penetratin, oligoarginine), amphipatic (e.g., transportin, TP10, Pep-1), hydrophobic (e.g., azurin, crotamine, maurocalcine, lycosin-1), or chimeric (transportan). CPPs include the YARA peptides disclosed herein as well as other known CPPs, such as those described by, and incorporated by reference to Reissmann, S. et al., New generation of cell-penetrating peptides: functionality and potential clinical application, J. PEPTIDE SCI. 2021, 27 (5), J. Xie, et al, Cell-penetrating peptides in diagnosis and treatment of human diseases: from preclinical research to clinical application, FRONT. PHARMCOL., 2020, 11; or H. Derakhshankhab, et al., Cell penetrating peptides: a concise review with emphasis on biomedical applications. BIOMEDICINE & PHARMACOTHERAPY, 2018, 108, 1090. A CPP may be bound or associated with the N-terminal or C-terminal of an active peptide, such as P35 or P50 or the other active peptides or modified peptides disclosed herein.

Nucleic acids encoding the peptides or peptide products described herein include vectors carrying a coding sequence for the peptides or peptide products disclosed herein, such as those encoding P35 or P50 peptides. In some embodiments, a nucleic acid or modified nucleic acid encoding a peptide of interest, such as P35 or P50, may be administered instead of a peptide. Typically, the nucleic acid will encode the peptide of interest once it is transported into the cell. Vectors that can transform a target cell such as a neuron with a sequence encoding a peptide as disclosed herein such as P35 or P50 include plasmid-based expression vectors, viral expression vectors such as adeno associated virus, herpes simplex virus, or lentiviruses. Such vectors may be delivered to target cells in combination with CPPs, lipid nanoparticles, or synthetic cationic polymers.

Modes of administration. The peptides and peptide-related products described herein may be administered to a subject in need of prophylaxis against or treatment for a tauopathy may be administered by any route that contacts the peptide or peptide-related product with nervous system tissue such as neurons. These include systemic administration, direct intranasal administration for example to target a nose-to-brain pathway bypassing the BBB which may optionally be in combination with chitosan or another absorption enhancer, administration in combination with a cell penetrating peptide or other transporter molecules that facilitate neuronal uptake, administration in an encapsulated form, such as in a liposome or nanoparticle, administration in a form that provides receptor-mediated transcytosis across the BBB, administering targeting axonal import (TAxl) using peptides that deliver payloads to specific neuronal tissues. These are other modes of administration are described by and incorporated by reference to Salameh, T. S. et al, Delivery of therapeutic peptides and proteins to the CNS, ADV. PHARMACOL, 2014, 71, 277; as described by and incorporated by reference to Lalatsa, et al., Strategies to deliver peptide drugs to the brain, MOL. PHARMACEUTICS, 2014, 11 (4), 1081; or as described by M. H. Baig, et al., Peptide based therapeutics and their use for the treatment of neurodegenerative and other diseases, BIOMED. & PHARMACOTHERAPY, 2018, 103, 574. The peptides, peptide products, and nucleic acids disclosed herein that encode peptides such as P3S or P50 may be administered in vivo, ex vivo, or in vitro.

Functional effects of administering P35, P50 or other selected peptide fragments of FKBP52 include prevention of FKBP52 decrease, increases in free FKBP52, inhibition of interaction between FKBP52 and Tau proteins, blockade of Tau aggregation.

Peptides of interest are able to interact with Tau proteins. These peptides likely inhibit Tau aggregation by interacting with the polymerizing side of the Tau aggregates.

The present invention comprises, but is not limited to, the following embodiments:
1. Embodiment 1: An embodiment of the present invention is directed to a method for preventing, reducing the severity of, or treating a tauopathy comprising administering to a subject in need thereof a peptide fragment of FK506-binding protein (FKBP52) or a modified form thereof.
2. Embodiment 2: concerns a method of embodiment 1, wherein the tauopathy is Alzheimer's Disease (AD), familial FTLD-Tau or progressive supranuclear palsy (PSP)
3. Embodiment 3: concerns a method of embodiment 1 or 2, wherein the peptide fragment consists of P35 (SEQ ID NO: 1) or a fragment thereof.
4. Embodiment 4: concerns a method of embodiment 1, 2, or 3, wherein the peptide fragment consists of a modified form of P35 (SEQ ID NO: 1) having backbone protection or a modified N or C terminus.
5. Embodiment 5; concerns a method of embodiment 1, 2, 3, or 4, wherein the peptide fragment consists of a modified form of P35 (SEQ ID NO:1) that further comprises a cell penetrating peptide linked to P35.
6. Embodiment 6: concerns a method of embodiment 1, 2, 3, 4, or 5, wherein the peptide fragment consists of a modified form of P35 (SEQ ID NO: 1) that further comprises cell penetrating peptide YARAAARQARA (SEQ ID NO: 3).
7. Embodiment 7: concerns a method of embodiment 1, 2, 3, 4, 5, or 6, wherein the peptide fragment consists of P50 (SEQ ID NO: 2) or a fragment thereof.
8. Embodiment 8: concerns a method of embodiment 1, 2, 3, 4, 5, 6 or 7, wherein the peptide fragment consists of a modified form of P50 (SEQ ID NO:2) having backbone protection or a modified N or C terminus.
9. Embodiment 9: concerns a method of embodiment 1, 2, 3, 4, 5, 6, 7 or 8, wherein the peptide fragment consists of a modified form of P50 (SEQ ID NO: 2) that further comprises a cell penetrating peptide linked to P50.
10. Embodiment 10: concerns a method of embodiment 1, 2, 3, 4, 5, 6, 7, 8 or 9, wherein the peptide fragment consists of a modified form of P50 (SEQ ID NO: 2) that further comprises cell penetrating peptide YARAAARQARA (SEQ ID NO: 3).
11. Embodiment 11: concerns a peptide or modified peptide or active peptide comprising a fragment of FKBP52 that inhibits Tau protein aggregation.
12. Embodiment 12: concerns a peptide or modified peptide of Embodiment 11 that is P35 or P50.
13. Embodiment 13: concerns a modified peptide of Embodiment 11 or 12 that has a modified backbone or a modified C or N terminus.
14. Embodiment 14 concerns a modified peptide of Embodiment 11, 12, or 13 that is CP35 or CP50 that has an N terminal cysteine residue.
15. Embodiment 15 concerns a modified peptide of Embodiment 11, 12, 13, or 14 that further comprises a cell penetrating peptide.
16. Embodiment 16 concerns a modified peptide of Embodiment 11, 12, 13, 14, or 15 that further comprises cell penetrating peptide YARAAARQARA (SEQ ID NO: 3).
17. Embodiment 17 concerns a peptide or modified peptide according to at least one of, or any one of, Embodiments 11 to 16, wherein the active amino-acid sequence comprises at least one VY sequence and less than 30 amino acids.
18. Embodiment 18 concerns a pharmaceutical composition comprising at least one active peptide or modified peptide according to at least one of, or any one of Embodiments 11 to 17 in association with a non-replicative adenoviral vector or with a non-replicative retroviral vector in a form suitable for penetrating a cell of the nervous system, such as a neuron, and expressing the active or modified peptide.
19. Embodiment 19 concerns an in vitro detection or diagnostic kit comprising a labelled detection system to identify the delivery of an active peptide into a cell of the nervous system, such as a neuron, of a subject and to visualize the biological complex formed between Tau protein or a part of said protein with an active peptide according any one of Embodiments 11 to 17.

The above written description of the invention provides a manner and process of making and using it such that any person skilled in this art is enabled to make and use the same, this enablement being provided in particular for the subject matter of the appended claims, which make up a part of the original description. The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. Having generally described this invention, a further understanding can be obtained by reference to certain specific examples or data or technical information, which are provided herein for purposes of illustration only, and are not intended to be limiting unless otherwise specified.

1. A method for preventing, reducing the severity of, or treating a tauopathy comprising administering to a subject in need thereof a peptide fragment of FK506-binding protein (FKBP52) or a modified form thereof.

2. The method of claim 1, wherein the tauopathy is Alzheimer's Disease (AD), familial FTLD-Tau, progressive supranuclear palsy (PSP) or other tauopathies.

3. The method of claim 1, wherein the peptide fragment consists of P35 (SEQ ID NO: 1).

4. The method of claim 1, wherein the peptide fragment consists of a modified form of P35 (SEQ ID NO: 1) having backbone protection or a modified N or C terminus.

5. The method of claim 1, wherein the peptide fragment consists of a modified form of P35 (SEQ ID NO: 1) that further comprises a cell penetrating peptide linked to P35.

6. The method of claim 1, wherein the peptide fragment consists of a modified form of P35 (SEQ ID NO: 1) that further comprises cell penetrating peptide YARAAARQARA (SEQ ID NO: 3).

7. The method of claim 1, wherein the peptide fragment consists of P50 (SEQ ID NO: 2).

8. The method of claim 1, wherein the peptide fragment consists of a modified form of P50 (SEQ ID NO: 2) having backbone protection or a modified N or C terminus.

9. The method of claim 1, wherein the peptide fragment consists of a modified form of P50 (SEQ ID NO: 2) that further comprises a cell penetrating peptide linked to P50.

10. The method of claim 1, wherein the peptide fragment consists of a modified form of P50 (SEQ ID NO: 2) that further comprises cell penetrating peptide YARAAARQARA (SEQ ID NO: 3).

11. A peptide or modified peptide comprising a fragment of FKBP52 that inhibits Tau protein aggregation.

12. The peptide or modified peptide of claim 11 that is P35 or P50.

13. The modified peptide of claim 11 that has a modified backbone or a modified C or N terminus.

14. The modified peptide of claim 11 that is CP35 or CP50 that has an N terminal cysteine residue.

15. The modified peptide of claim 11 that further comprises a cell penetrating peptide.

16. The modified peptide of claim 11 that further comprises cell penetrating peptide YARAAARQARA (SEQ ID NO: 3).

17. A peptide or modified peptide according to claim 11, wherein the active amino-acid sequence comprises at least one VY sequence and less than 30 amino acids

18. A pharmaceutical composition comprising in the active according to at least one active peptide or modified peptide of claim 11 associated with a non replicative adenoviral vector or with a non-replicative retroviral vector penetrating in neurons cells and expressing the said peptide.

19. An in vitro diagnostic kit comprising a labelled detection system to identify the delivery of the active peptide in neurons of a subject and to visualize the biological complex formed between the Tau protein or a part of said protein with an active peptide according to claim.


SEQUENCE LISTING (12 sequences)

SEQ ID NO: 1 - protein, 35 aa, Homo sapiens
AKPNEGAIVEVALEGYYKDKLFDQRELRFEIGEGE

SEQ ID NO: 2 - protein, 50 aa, Homo sapiens
AKPNEGAIVEVALEGYYKDKLFDQRELRFEIGEGENLDLPYGLERAIQRM

SEQ ID NO: 3 - protein, 11 aa, synthetic construct
YARAAARQARA

SEQ ID NO: 4 - protein, 35 aa, synthetic construct
IRFKVLEGLYGEVDEAKEGNEAKAEGLQEYPDFIR

SEQ ID NO: 5 - protein, 50 aa, synthetic construct
AKGNVEAIVEPALEGYDKYKLDFQRELREFIGELGNLEDLYGQPARIERM

SEQ ID NO: 6 - protein, 51 aa, unidentified
CAKPNEGAIVEVALEGYYKDKLFDQRELRFEIGEGENLDLPYGLERAIQRM

SEQ ID NO: 7 - protein, 36 aa, synthetic construct
CAKPNEGAIVEVALEGYYKDKLFDQRELRFEIGEGE

SEQ ID NO: 8 - protein, 61 aa, synthetic construct
YARAAARQARAAKPNEGAIVEVALEGYYKDKLFDQRELRFEIGEGENLDLPYGLERAIQRM

SEQ ID NO: 9 - protein, 46 aa, Synthetic construct
YARAAARQARAAKPNEGAIVEVALEGYYKDKLFDQRELRFEIGEGE

SEQ ID NO: 10 - protein, 10 aa, synthetic construct
  MOD_RES 1: note = X = modified cysteine residue having N-terminal acetylation and a 3-nitro-2-pyridinesulfenyl (Npys) protecting group on the thiol side of the cysteine
XRRRRRRRRR

SEQ ID NO: 11 - protein, 12 aa, synthetic construct
  MOD_RES 1: note = X = modified cysteine residue having N-terminal acetylation and a 3-nitro-2-pyridinesulfenyl (Npys) protecting group on the thiol side of the cysteine
XYARAAARQARA

SEQ ID NO: 12 - protein, 6 aa, synthetic construct
VQIVYK